(19) United States
(12) Patent Application Publication
(10) Pub. No.: US 2025/0331790 A1
(43) Pub. Date: Oct. 30, 2025

(54) METHODS AND SYSTEMS FOR AN INTEGRATED FILTER SYSTEM WITH TWO CARRIAGES

(71) Applicant: GE Precision Healthcare LLC, Waukesha, WI (US)

(72) Inventors: Brandon Smith, Waukesha, WI (US); Dominic Crotty, Cork (IE); Chang Lyong Kim, Brookfield, WI (US); Timothy Matthew Belmer, Milwaukee, WI (US)

(21) Appl. No.: 19/255,512

(22) Filed: Jun. 30, 2025

Related U.S. Application Data
(63) Continuation-in-part of application No. 17/828,928, filed on May 31, 2022, now Pat. No. 12,369,869.

(51) Int. Cl. A61B 6/40; A61B 6/58

(52) U.S. Cl. CPC A61B 6/405; A61B 6/4035; A61B 6/582

(57) ABSTRACT

Various methods and systems are provided for integrated filter assemblies including a bowtie filter and a hardening filter mounted on a single carriage. In one embodiment, an imaging system may include an x-ray source to generate an x-ray beam, a pre-patient collimator positioned adjacent to the x-ray source such that the x-ray beam passes through the pre-patient collimator. The pre-patient collimator includes a carriage, and at least one filter coupled to one edge of the carriage and extending away from the carriage.

### CROSS-REFERENCE TO RELATED APPLICATIONS

This application is a continuation-in-part of U.S. application Ser. No. 17/828,928, filed on May 31, 2022, the disclosure of which is incorporated herein by reference in its entirety.

### FIELD

Embodiments of the subject matter disclosed herein relate to diagnostic medical imaging, and more particularly, to computed tomography imaging setup with an integrated filter assembly.

### BACKGROUND

Noninvasive imaging modalities may transmit energy in the form of radiation into an imaging subject. Based on the transmitted energy, images may be subsequently generated indicative of the structural or functional information internal to the imaging subject. In computed tomography (CT) imaging, radiation transmits from a radiation source to a detector through the imaging subject. A bowtie filter may be positioned between the radiation source and the imaging subject for adjusting the spatial distribution of the radiation energy based on the anatomy of the imaging subject. The bowtie filter may be designed to distribute higher radiation energy to specific imaging region of the subject. As a result, the quality amplitude of signal received by the imaging detector is improved in the central area, and the radiation dose on the periphery of the specific imaging subject is reduced. Different anatomy of the subject may require different bowtie filters. For example, bowtie filters of different shape and size may be designed to image distinct regions of the subject's body such as the head, the chest, and the abdomen.

Further, a hardening filter may be positioned between the radiation source and the imaging subject for intercepting the lower energy radiations, thereby attenuating and “hardening” the beam. Conditioning of the beam via a hardening filter may be specifically desired during calibration or during a diagnostic patient scan or a scout scan which may precede a diagnostic scan and may provide a projection view along a longitudinal axis of the subject including the internal structure of the subject. Therefore, a setup for integrating one or more bowtie filters and a hardening filter is needed.

### BRIEF DESCRIPTION

In one embodiment, an imaging system may include an x-ray source to generate an x-ray beam, a pre-patient collimator positioned adjacent to the x-ray source such that the x-ray beam passes through the pre-patient collimator. The pre-patient collimator includes a carriage, and at least one filter coupled to one edge of the carriage and extending away from the carriage.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a pictorial view of an imaging system according to an embodiment of the invention.

FIG. 2 shows a block schematic diagram of an exemplary imaging system according to an embodiment of the invention.

FIG. 3 shows a perspective view of an example integrated filter assembly including a carriage, a hardening filter, and a bowtie filter.

FIG. 4 shows a first partial exploded view of the example integrated filter assembly of FIG. 3.

FIG. 5 shows a second partial exploded view of the example integrated filter assembly of FIG. 3.

FIG. 6 shows a partial exploded view of a second example integrated filter assembly including a carriage and at least one bowtie filter.

FIGS. 7A and 7B depict cross-sectional views of the example integrated filter assembly of FIGS. 3-5 and the example integrated filter assembly of FIG. 6 positioned within the example imaging system of FIG. 1.

FIG. 8 depicts a cross-sectional view of an alternative configuration of the example integrated filter assembly of FIGS. 3-5 and the example integrated filter assembly of FIG. 6 positioned within the example imaging system of FIG. 1.

FIG. 9 depicts a cross-sectional view of another alternative configuration of the example integrated filter assembly of FIGS. 3-5 and the example integrated filter assembly of FIG. 6 positioned within the example imaging system of FIG. 1.

FIG. 10 depicts a cross-sectional view of another alternative configuration of the example integrated filter assembly of FIGS. 3-5 and the example integrated filter assembly of FIG. 6 positioned within the example imaging system of FIG. 1.

FIG. 11A shows a first position of a filter assembly with three bowtie filters and a hardening filter.

FIG. 11B shows a second position of the filter assembly of FIG. 11A.

FIG. 11C shows a third position of the filter assembly of FIG. 11A.

FIG. 11D shows a fourth position of the filter assembly of FIG. 11A.

FIG. 11E shows a fifth position of the filter assembly of FIG. 11A.

FIG. 11F shows a sixth position of the filter assembly of FIG. 11A.

FIG. 12A depicts a block diagram illustrating a scan using the example filter assembly in one of the second, third, or sixth positions depicted in FIGS. 11B, 11C, and 11F.

FIG. 12B depicts a block diagram illustrating a scan using the example filter assembly in the fifth position depicted in FIG. 11E.

FIG. 12C depicts a block diagram illustrating a scan using the example filter assembly in the fourth position depicted in FIG. 11D.

FIG. 13 shows a perspective view of another example integrated filter assembly including a carriage, hardening filters, and a bowtie filter.

FIG. 14 shows a perspective view of another example integrated filter assembly including a carriage and one or more bowtie filters.

FIG. 15 shows a perspective view of the example filter assembly carriages of FIGS. 13 and 14 positioned together, in one example position.

FIG. 16 shows a perspective view of another example integrated filter assembly including a carriage, hardening filters, and a bowtie filter.

FIG. 17A shows a first example position of the example filter assembly including the carriages depicted in FIGS. 13-15.

FIG. 17B shows a second example position of the example filter assembly including the carriage depicted in FIGS. 13 and 14.

FIG. 17C shows a third example position of the example filter assembly including the carriage depicted in FIGS. 13 and 14.

FIG. 17D shows a fourth example position of the example filter assembly including the carriage depicted in FIGS. 13 and 14.

FIG. 17E shows a fifth example position of the example filter assembly including the carriage depicted in FIGS. 13 and 14.

FIG. 17F shows a sixth example position of the example filter assembly including the carriage depicted in FIGS. 13 and 14.

FIG. 18 depicts example energy spectrum curves for different filters that may be used with the example filter assembly described herein.

FIG. 19 depicts a comparison between the energy spectrum of an unfiltered X-ray and a filtered X-ray.

FIG. 20 shows a flow chart of an example method for calibration using the integrated filter assembly.

FIG. 21 shows a flow chart of an example method for imaging using multiple filters included in the integrated filter assembly.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

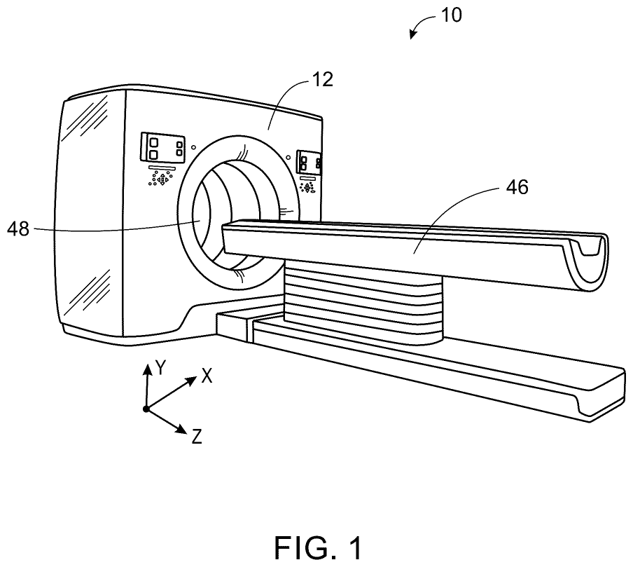

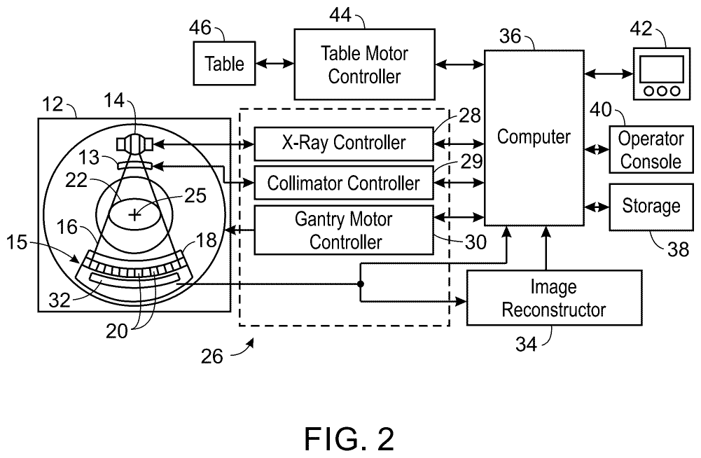

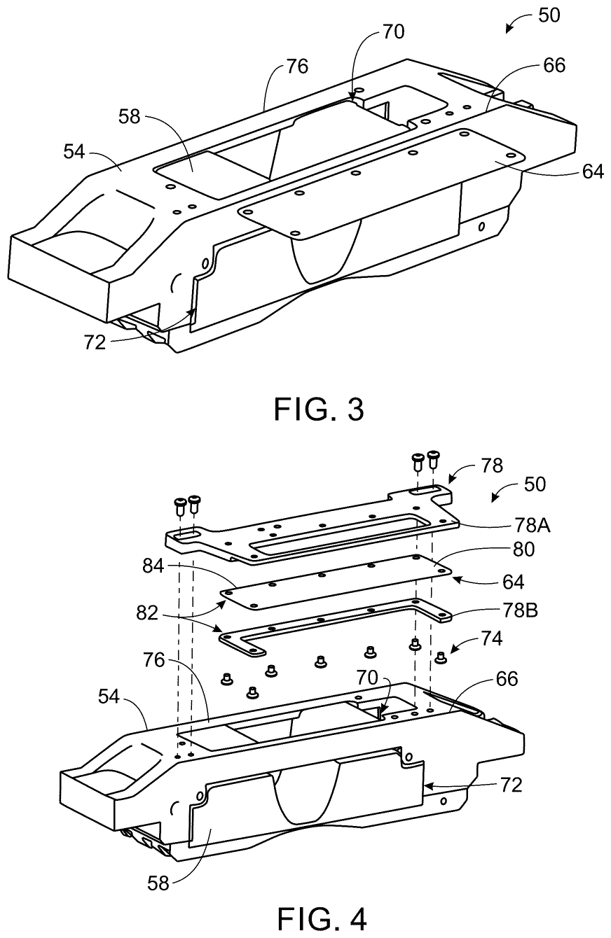

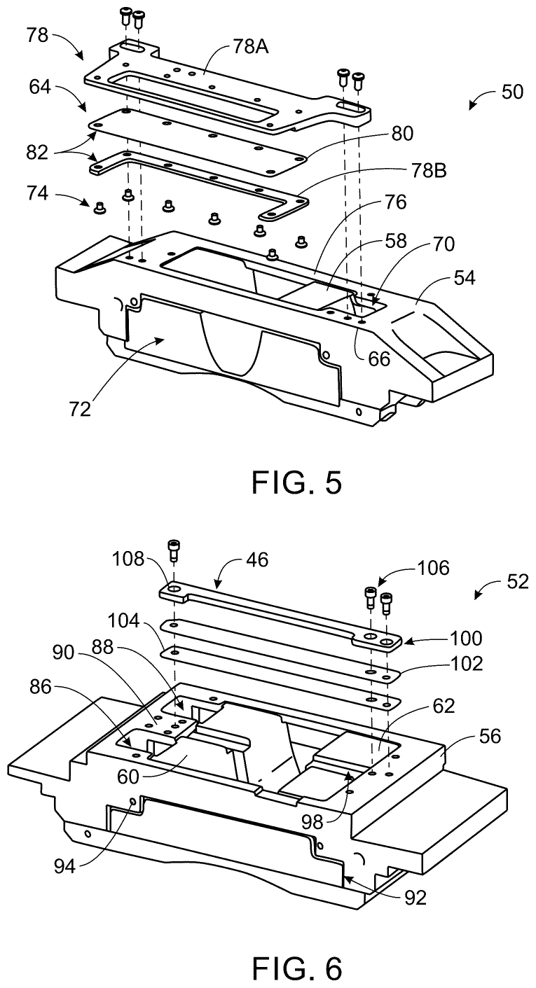

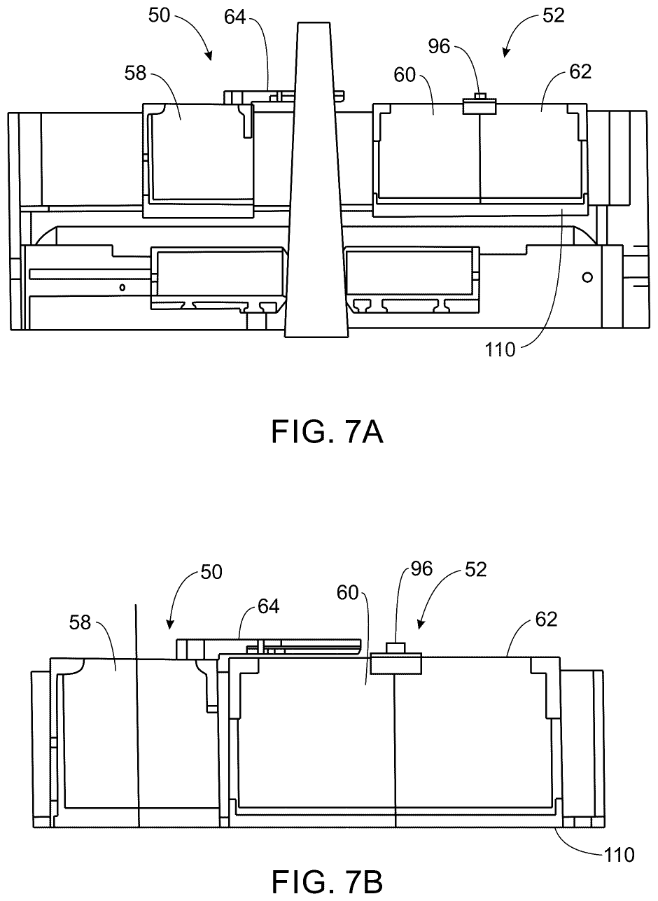

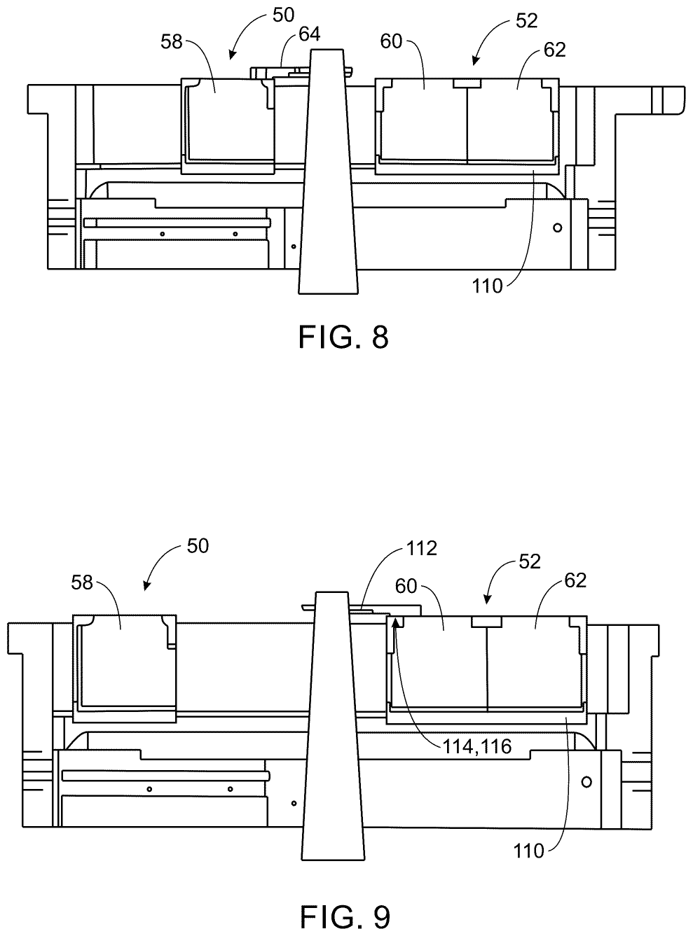

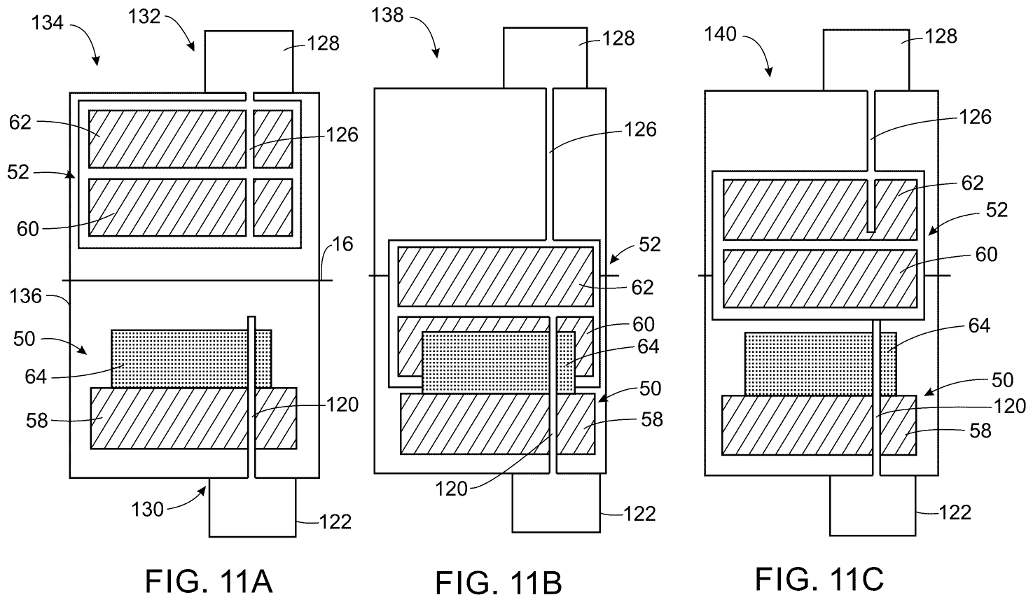

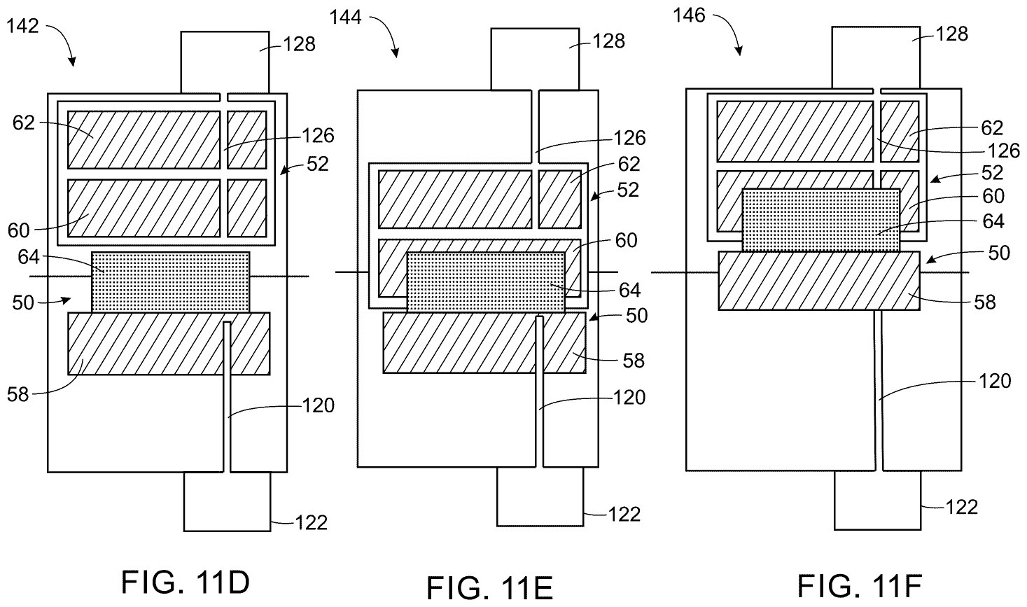

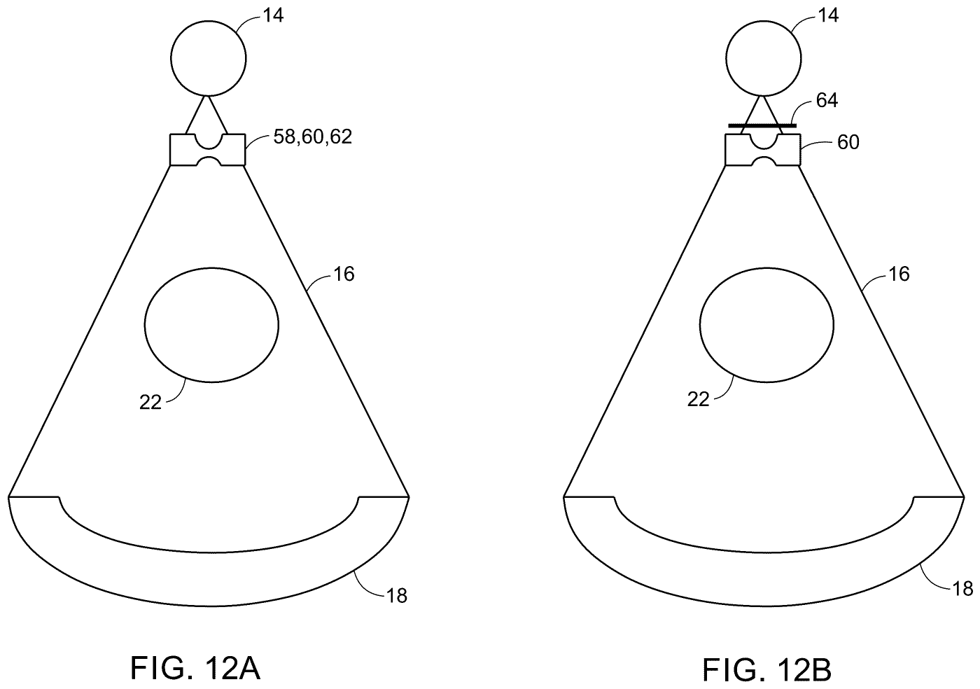

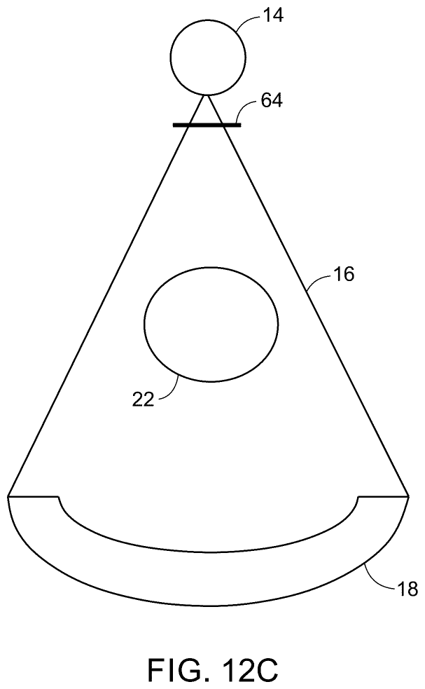

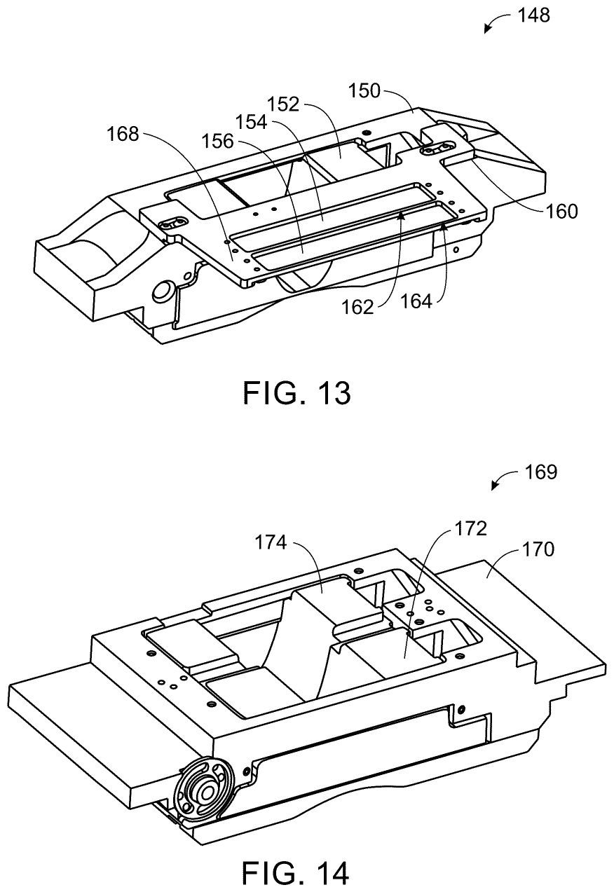

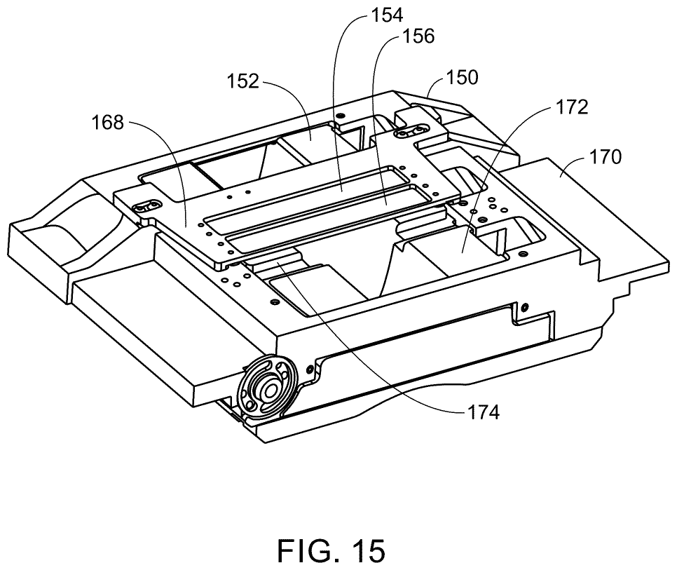

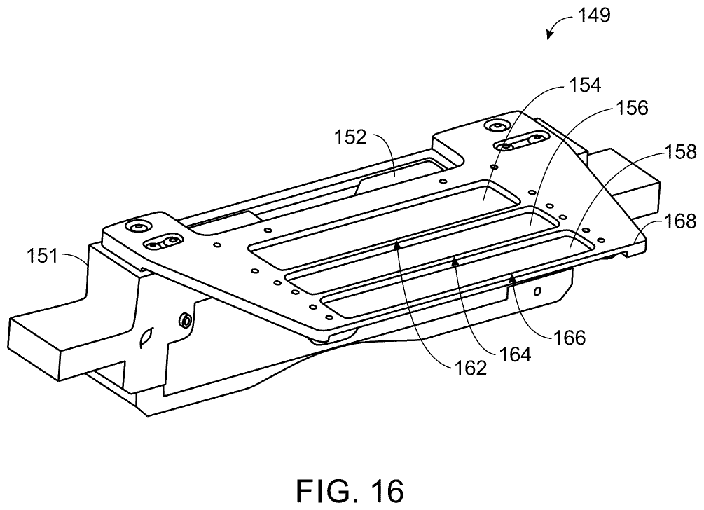

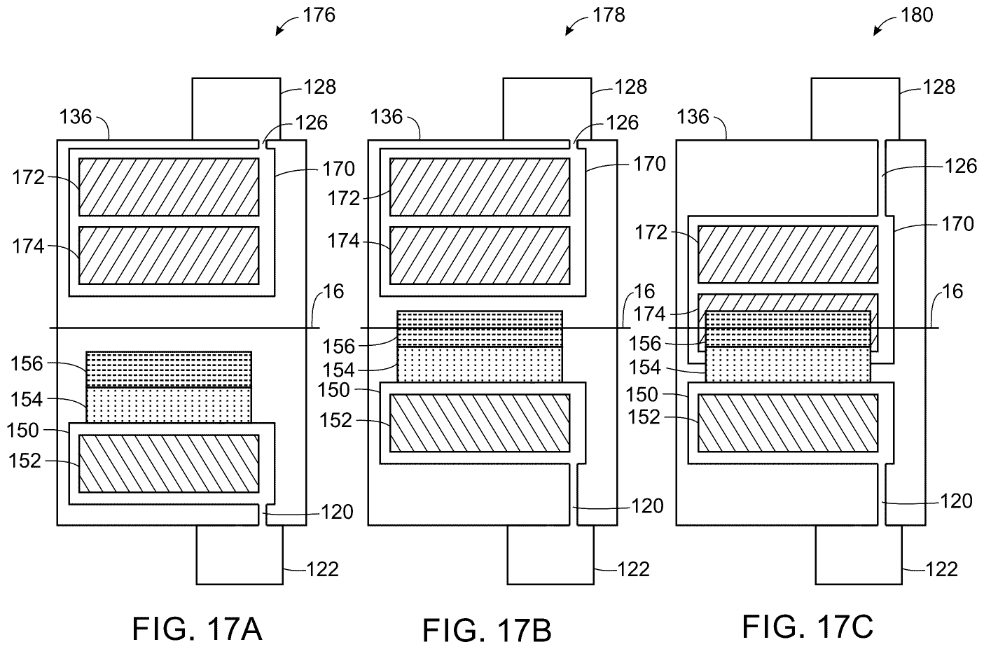

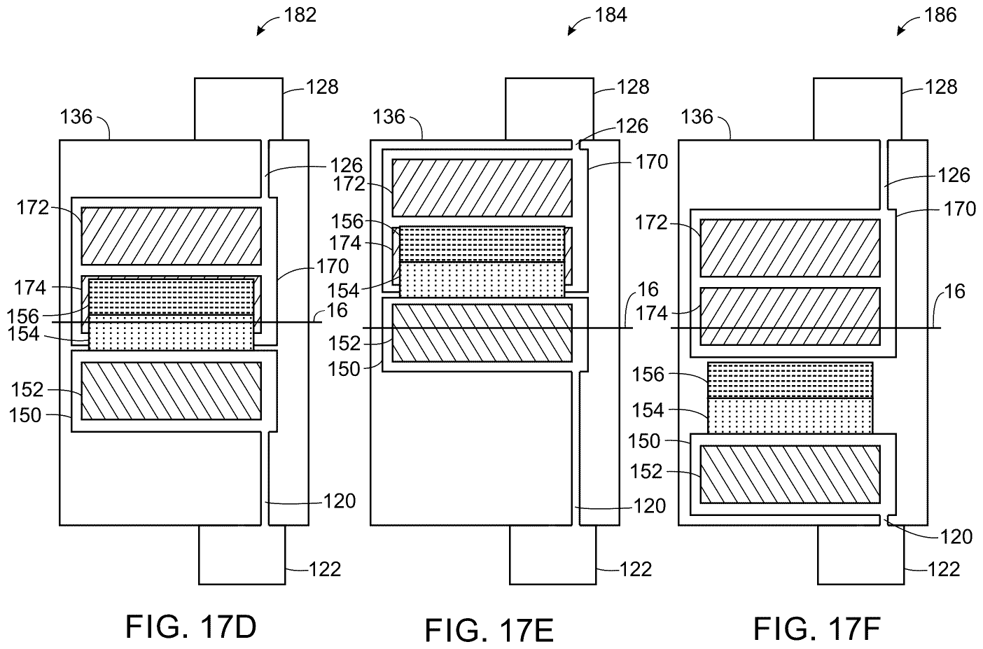

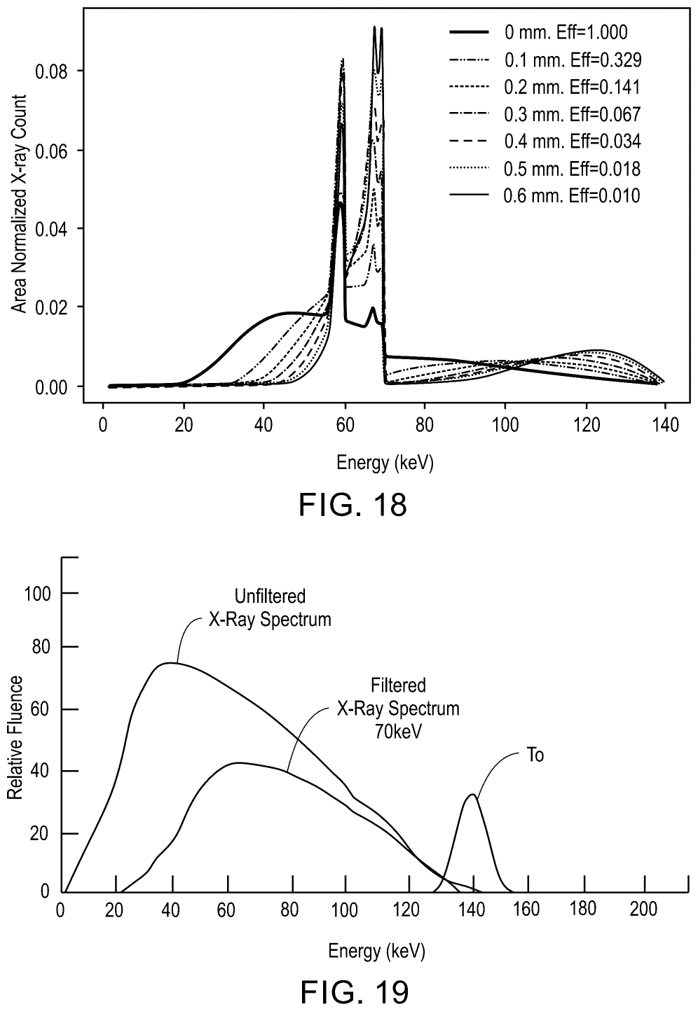

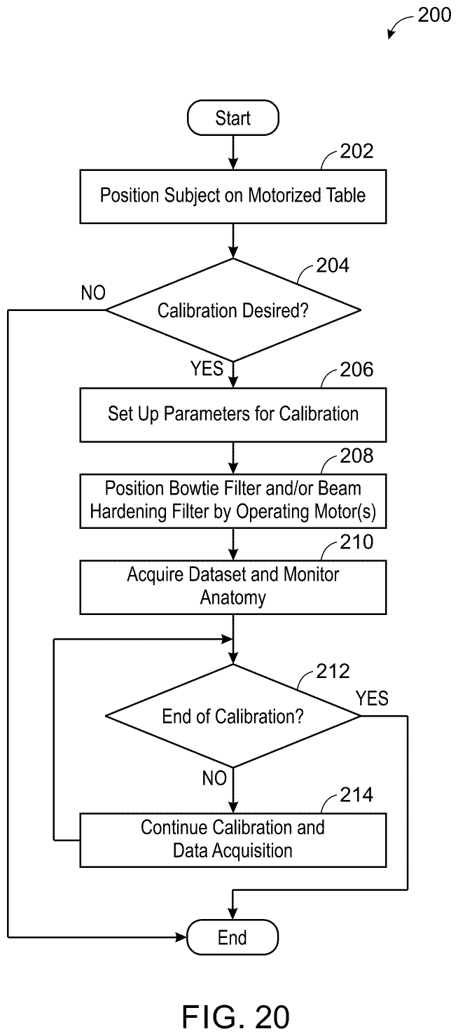

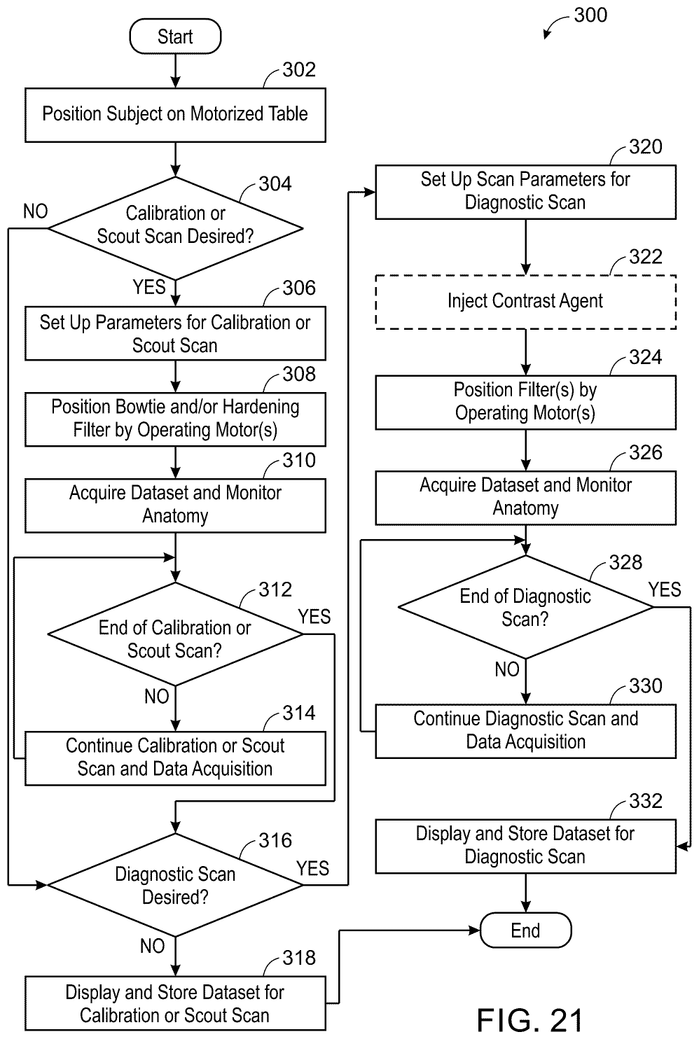

The following description relates to various embodiments of x-ray imaging of a subject. In particular, systems and methods are provided for CT imaging using one or more of a hardening filter and bowtie filters. FIGS. 1-2 show an example embodiment of an imaging system, wherein the one or more filters are positioned between the radiation source and the imaging subject. Different filters may be selected based on the anatomy of the imaging subject being imaged or need for calibration. FIGS. 3-5 shows an example of an integrated filter assembly including a carriage, a hardening filter, and a bowtie filter, which may be positioned to adjust a spatial distribution and condition the beam reaching the subject. As an example, in a single carriage, a bowtie filter may be positioned adjacent to a hardening filter that is coupled to one edge of the carriage. The bowtie filter or the hardening filter may be positioned in a path of the beam by moving the carriage along an axis perpendicular to the beam. FIG. 6 shows an example second carriage including two bowtie filters and an optional hardening filter that may be used in conjunction with the first carriage. The second carriage may be positioned such that a filter of the second carriage is positioned in the path of the beam simultaneously with the hardening filter of the first carriage. FIGS. 7A-7B show cross-sectional views of different positions of the example first and second carriages. FIGS. 8-10 depict different configurations of a first and second carriages including different hardening filter positions. For example, the hardening filter may be coupled to the second carriage (e.g., the carriage with two bowtic filters). FIGS. 11A-11F show various positions of an example filter assembly with three bowtie filters and a hardening filter. FIGS. 12A-12C are block diagrams representing various scans that may be implemented using the example filter assembly. For example, the hardening filter may be used for a low-dose scan and/or for calibration of the imaging system. FIG. 13 depicts an additional example of an integrated filter assembly including a carriage, hardening filters, and a bowtie filter. FIG. 14 depicts an additional example of a carriage including one or more bowtie filters, which may be used in conjunction with the example carriage of FIG. 13, or any of the other example carriages discussed herein. FIG. 15 depicts the carriages of FIGS. 13 and 14 in one example position of many possible positions. FIG. 16 depicts another example integrated filter assembly including a carriage, hardening filters and a bowtie filter. FIGS. 17A-F depict different positions of the additional example carriages of FIGS. 13 and 14. FIGS. 18 and 19 graphically represent the reduced dose and energy spectrum when using the example filter assembly. FIG. 20 shows an example method for calibrating an imaging system using one or more filters included in the integrated filter assembly. FIG. 21 shows an example method for imaging a subject using one or more filters included in the integrated filter assembly.

Though a CT system is described by way of example, it should be understood that the present techniques may also be useful when applied to images acquired using other imaging modalities, such as tomosynthesis, C-arm angiography, and so forth. The present discussion of a CT imaging modality is provided merely as an example of one suitable imaging modality.

Various embodiments may be implemented in connection with different types of imaging systems. For example, various embodiments may be implemented in connection with a CT imaging system in which a radiation source projects a fan- or cone-shaped beam that is collimated to lie within an x-y plane of a Cartesian coordinate system and generally referred to as an “imaging plane.” The x-ray beam passes through an imaging subject, such as a patient. The beam, after being attenuated by the imaging subject, impinges upon an array of radiation detectors. The intensity of the attenuated radiation beam received at the detector array is dependent upon the attenuation of an x-ray beam by the imaging subject. Each detector element of the array produces a separate electrical signal that is a measurement of the beam intensity at the detector location. The intensity measurements from all the detectors are acquired separately to produce a transmission profile.

In third-generation CT systems, the radiation source and the detector array are rotated with a gantry within the imaging plane and around an object (such as a region of the subject) to be imaged such that the angle at which the x-ray beam intersects the imaging subject constantly changes. A complete gantry rotation occurs when the gantry concludes one full 360-degree revolution. A group of x-ray attenuation measurements (e.g., projection data) from the detector array at one gantry angle is referred to as a “view.” A view is, therefore, each incremental position of the gantry. A “scan” of the object comprises a set of views made at different gantry angles, or view angles, during one revolution of the x-ray source and detector. In an axial diagnostic scan, the projection data is processed to construct an image that corresponds to a two-dimensional slice taken through the imaging subject. A scout scan (also referred herein as localizer scan) provides a projection view along a longitudinal axis of the imaging subject and generally provides aggregations each including internal structures of the subject. One method for reconstructing an image from a set of projection data is referred to in the art as a filtered back-projection technique. This process converts the attenuation measurements from a scan into integers called “CT numbers” or “Hounsfield units” (HU), which are used to control the brightness of a corresponding pixel on a display.

Beam characteristics such as size, shape, and energy may be different for a scout scan (also referred herein as localizer scan) and a diagnostic scan. During certain scout scans and diagnostic scans, it is desired to use a higher power x-ray source. The higher power improves the quality of the diagnostic scan and increases thermal stability of the x-ray tube including the target. However, an increase in the x-ray power, may increase in x-ray radiation exposure for a patient. The hardening filter may be used in the path of the beam to attenuate the beam and reduce the amount of the lower energy x-ray beam prior to it entering the patient's body. Different materials may be used for hardening filters to perform different functions, and thus multiple hardening filters may be included on the filter assembly. For example, a first hardening filter may be used for calibrations, while a second hardening filter may be used for scout scans. In some examples, an additional hardening filter may be used for other types of scout and/or diagnostic scans. One of the example hardening filter along with an example bowtie filter may be used when a higher energy x-ray beam is desired for a patient scan, for example, a large patient. The hardening filter and the bowtie filters may be mounted on separate carriages which can be moved in and out of the beam as desired. However, adding multiple carriages will add cost and complexity to the apparatus. Also, the time to complete scans may be longer due to the need to move carriages in and out of the beam between sections of a scan. Therefore, according to embodiments disclosed herein, a single integrated filter assembly may in incorporated including a carriage, a plurality of hardening filters, and a plurality of bowtie filters. Based on the scan setup, one or more filters from the carriage may be placed in the path of the beam. By including multiple bowtie and hardening filters in a single integrated filter assembly, reliability of the set up may be increased while cost and complexity of the setup may be decreased.

FIG. 1 illustrates an exemplary computed tomography (CT) imaging system 10 and FIG. 2 depicts an example block diagram of the exemplary imaging system according to an embodiment of the invention. The CT imaging system includes a gantry 12. The gantry 12 has an X-ray source 14 that generates and projects a beam of X-rays 16 toward a detector assembly 15 on the opposite side of the gantry 12. The X-ray source 14 projects the beam of X-rays 16 through a pre-patient collimator assembly 13 that conditions the beam of X-rays 16 using, for example, one or more filters. The detector assembly 15 includes a collimator assembly 18 (a post-patient collimator assembly), a plurality of detector modules 20 (e.g., detector elements or sensors), and data acquisition systems (DAS) 32. The plurality of detector modules 20 detect the projected X-rays that pass through a subject or object 22 being imaged, and DAS 32 converts the data into digital signals for subsequent processing. Each detector module 20 in a conventional system produces an analog electrical signal that represents the intensity of an incident X-ray beam and hence the attenuated beam as it passes through the subject or object 22. During a scan to acquire X-ray projection data, gantry 12 and the components mounted thereon rotate about a center of rotation 25 (e.g., isocenter) so as to collect attenuation data from a plurality of view angles relative to the imaged volume.

Rotation of gantry 12 and the operation of X-ray source 14 are governed by a control system 26 of CT imaging system 10. Control system 26 includes an X-ray controller 28 that provides power and timing signals to an X-ray source 14, a collimator controller 29 that controls a length and a width of an aperture of the pre-patient collimator 13 (and, thus, the size and shape of the beam of X-rays (e.g., x-ray beam) 16), and a gantry motor controller 30 that controls the rotational speed and position of gantry 12. An image reconstructor 34 receives sampled and digitized X-ray data from DAS 32 and performs high-speed image reconstruction. The reconstructed image is applied as an input to a computer 36, which stores the image in a storage device 38. Computer 36 also receives commands and scanning parameters from an operator via console 40. An associated display 42 allows the operator to observe the reconstructed image and other data from computer 36. The operator supplied commands and parameters are used by computer 36 to provide control signals and information to DAS 32, X-ray controller 28, collimator controller 29, and gantry motor controller 30. In addition, computer 36 operates a table motor controller 44, which controls a motorized table 46 to position subject 22 and gantry 12. Particularly, table 46 moves portions of subject 22 through a gantry opening or bore 48.

In accordance with aspects of the present disclosure, the imaging system 10 is configured to perform automatic exposure control responsive to user input. Exposure control may be achieved using one or more filter assemblies (e.g., filter assemblies 50 and 52 of FIGS. 3-6) that may be mounted within gantry 12 between x-ray source 14 and the subject 22. The filter assemblies 50, 52 may travel in and out of the beam 16 in the z-direction while the beam 16 is substantially in the y-direction. In the example described herein, the filter assemblies 50, 52 include multiple bowtie filters and at least one hardening filter. The assemblies 50, 52 may be positioned such that more than one filter may be positioned in the path of the x-ray beam 16 during a scan.

FIGS. 3-6 depict example filter assemblies 50, 52 that may be used with the example CT imaging system 10 described herein. The two filter assemblies 50, 52 may each include a carriage 54, 56, and each carriage 54, 56 includes at least one bowtie filter. Combined, the filter assemblies 50, 52 include multiple different bowtie filters. In particular, the first filter assembly 50 depicted in FIGS. 3-5 includes a first carriage 54 having a first bowtie filter 58, and the second filter assembly 52 depicted in FIG. 6 includes a second carriage 56 having a second bowtie filter 60 and a third bowtie filter 62. Additionally, the example carriages 54, 56 may include a hardening filter. In the illustrated examples of FIGS. 3-6, the first carriage 54 includes a first hardening filter 64 that extends from a top edge 66 of the carriage 54 and the second carriage 56 includes a second hardening filter 96 positioned between the second and third bowtie filters 60, 62. However, the hardening filters 64, 96 may be configured differently (e.g., as depicted in alternative constructions in FIGS. 8-10). The bowtie filters 58, 60, 62 are shown here in rectangular shape as an example. Each and every bowtie filter 58, 60, 62 is rigid and non-deformable. The bowtie filters 58, 60, 62 may alternatively have different shapes and material constructions to provide proper x-ray special spectrum for imaging various types of anatomies. The bowtie filter(s) 58, 60, 62 may change the spatial distribution of the radiation beam (i.e., condition the beam) in the axial plane of the imaging subject 22 (such as a patient). For example, the re-distributed radiation beam 16 may have higher energy at the center and lower energy at the periphery of the subject 22. Each of the bowtie filters 58, 60, 62 may be designed to image a specific anatomy or section of the human body, such as head, chest, and abdomen. During imaging, one of the bowtie filters 58, 60, 62 may be selected based on the anatomy of the subject 22 to be scanned, and the selected filter may be placed into the radiation beam path 16. Responsive to a change in the anatomy, the filter(s) may be changed from one to another. Based on a nature of the scan, the carriage(s) may be positioned such that a hardening filter may or may not be placed in the radiation beam path 16. The hardening filter(s) may attenuate the beam 16 and remove low energy components thereby conditioning the beam 16 for specific scans, such as a patient scan or a calibration.

The first filter assembly 50 is shown in FIGS. 3-5. The first filter assembly 50 may include a first rectangular carriage 54. The first carriage 54 may include a first slot 70 formed lengthwise within a cavity of the first carriage 54. In one example, the first slot 70 may extend through the entire length of the first carriage 54. In another example, the first slot 70 may partially extend through the length of the first carriage 54.

A first bowtie filter 58 may be housed within the first slot 70. The first bowtie filter 58 may be shaped as a “bowtie” with a first, straight long side and a second, parallel long side including a central ridge. The first bowtie filter 58 may be formed of graphite. A bowtie filter 58 may be used to adjust spatial distribution of an x-ray beam 16 passing through the filter 58 and the size of a bowtie filter 58 governs a level of spatial distribution adjustments made to the x-ray beam 16 passing through the filter 58. The carriage 54 may include cut-outs 72 on side wall through which a bowtie filter 58 may be visible. The bowtie filter 58 may be secured inside the slot 70 via nuts and bolts 94.

A hardening filter 64 may be coupled to the carriage 54 on an edge 66 of a top surface 76 of the carriage 54. The hardening filter 64 may be positioned adjacent to the first bowtie filter 58 and extends away from first bowtie filter 58 and from the edge 66 of the carriage 54. The physical sizing of the hardening filter 64 may vary, but may be equal to or smaller than the physical size of the filter 58 due to the proximity of the hardening filter 64 which is closer to the x-ray source 14 than filter 58. As the rectangular hardening filter 64 is positioned extending from a top surface 76 of the first carriage 54, the first and second carriages 54, 56 may be positioned such that the hardening filter 64 overlaps (e.g., extends over) a bowtie filter (e.g., the second bowtie filter 60 or the third bowtie filter 62) in the second carriage 56.

The hardening filter 64 may include a support structure 78, and one or more metallic sheets 80 underneath the support structure 78. In this example, the support structure 78 includes a window frame structure 78A and an aluminum support structure 78B on either side of the metallic sheet(s) 80. Each metallic sheet 80 and the support structure 78 may be stacked together and bolted at the edge 66 of the carriage 54 via a plurality of bolts 74. In this example, a plurality of concentric holes 82 are formed on an edge 84 of the first metallic sheet 80 and the support structure 78, and each bolt 74 (used to attach the layers of the hardening filter 64 to the carriage 54) may pass through each of the concentric holes 82 present in each layer. In one example, the support structure 78 may be made of a metal such as aluminum, and the metallic sheet 80 may be made of a same metal or different metals. Copper may alternatively be used to form the first metallic sheet 80 that filters low energy x-rays out and keeps high energy x-rays as shown in FIG. 14. Alternatively, when a specific energy threshold is necessary in the x-ray detector, the hardening filter 64 may be tungsten and used primarily for calibrating the imaging system 64. For example, photon counting CT has two or more energy thresholds to allocate each captured x-ray to two or more energy bins, for example, low and high energy bins. The energy binning enables material identification in patient images that enhances CT diagnostic capability. The calibration of energy thresholds in CT detector can be achieved by a mono energy x-ray source. While a thin tungsten film (<50 um) can act as a typical beam hardening filter, like Cu and Al, of FIG. 14, a thick tungsten of 200 um˜600 um absorbs most of polyenergetic x-ray tube output and emits tungsten specific K-xray of 69.5 keV. Other heavy elements, like Pb, emit their own specific K-xray energy and can be used as a mono energy source as well. 300 μm thick tungsten prevents more than 99% of the x-ray beam from penetrating the filter, resulting in a single energy x-ray at the specific energy of 69.5 keV (FIG. 13). The specific energy that emitted from a tungsten hardening filter is ideal for calibrating the energy thresholds for a photon counting imaging system.

The hardening filter 64 may be used to intercept lower energy radiation, thereby attenuating and “hardening” the x-ray beam 16 passing through the hardening filter 64. The degree of beam attenuation may depend on one or more of a number of attenuation layers (such as metallic sheets), the thickness of each attenuation layer, the materials used in the attenuation layers, and the overall size of the attenuation layers.

As an example, when using thinner or weaker sheets of hardening material in 64, the support plate 78 may be used to limit deflection of the hardening filter 64 due to gantry rotational forces which may act to bend the middle of the hardening material. In this embodiment, the support plate 78 is positioned outside of the cross-sectional area of the hardened x-ray beam 16 that is used for imaging. In this way, the hardening filter 64 may be solely accounted for in hardening the imaging x-ray beam 16 while being mechanically strengthened by the support plate 78 proximal to the area where the imaging beam 16 passes through the hardening filter 64. Furthermore, the support plate 78 may be made from a stiff but lightweight material such as aluminum to minimize excess x-ray scatter near the hardening filter 64. The first filter assembly 50 may be used with the example system 10 alone, or may be used with an additional filter assembly, such as the second filter assembly 52 of FIG. 6.

The second filter assembly 52 is shown in FIG. 6. The second filter assembly 52 may include a second rectangular carriage 56. The second carriage 56 may include a first slot 86 and a second slot 88 formed lengthwise within a cavity of the carriage 56. The first slot 86 may be separated from the second slot 88 via a tab 90. In one example, each of the two slots 86 and 88 may extend through the entire length of the second carriage 56. In another example, each of the two slots 86 and 88 may partially extend through the length of the carriage 56.

A second bowtie filter 60 may be housed within the first slot 86 while a third bowtie filter 62 may be housed in the second slot 88. In one example, the second bowtie filter 60 and the third bowtie filter 62 may be positioned next to each other but not in contact. In another example, the second bowtie filter 60 and the third bowtie filter 62 may be positioned next to each other in face-sharing contact. Each of the second bowtie filter 60 and the third bowtie filter 62 may be shaped as a “bowtie” with a first, straight long side and a second, parallel long side including a central ridge. In one example, the second bowtie filter 60 and the third bowtie filter 62 may be of the same size (such as width, length, thickness, etc.) In another example, the second bowtie filter 60 and the third bowtie filter 62 may be of different sizes (such as width, length, thickness). Each of the second bowtie filter 60 and the third bowtie-filter 62 may be formed of graphite. A bowtie filter may be used to adjust spatial distribution of an x-ray beam 16 passing through the filter and the size of a bowtie filter governs a level of spatial distribution adjustments made to the x-ray beam 16 passing through the filter. The carriage 56 may include cut-outs 92 on a side wall through which a bowtie filter 60, 62 may be visible. As shown in this example, the third bowtie filter 62 may be co-planer with a side wall and cut-out 92 of the carriage 56. The bowtie filters 60, 62 may be secured inside their respective slots 86, 88 via nuts and bolts 94.

A hardening filter 96 may be coupled to the carriage 56 between the second bowtie filter 60 and the third bowtie filter 62. The hardening filter 96 may be embedded in a recess 98 between the second bowtie filter 60 and the third bowtie filter 62. The length of the hardening filter 96 may be higher than or equal to the length of each of the second bowtie filter 60 and the third bowtie filter 62. However, the width of the hardening filter 96 may be narrower than the width of each of the second bowtie filter 60 and the third bowtie filter 62. As the rectangular hardening filter 96 is positioned between the second bowtie filter 60 and the third bowtie filter 62, the hardening filter 96 may at least partly overlap with each of the second bowtie filter 60 and the third bowtie filter 62 and may be in face sharing contact with the top/side surfaces of the bowtie filters.

The hardening filter 96 may include a support structure 100, and one or more metallic sheets 102, 104 underneath the support structure 100. In this example, a first metallic sheet 102 and a second metallic sheet 104 may be positioned under the support structure 100. Each of the first metallic sheet 102, the second metallic sheet 104, and the support structure 100 may be stacked together and bolted at each end to the carriage 56 via a plurality of bolts 106. In this example, a plurality of concentric holes 108 are formed on two ends of each of the first metallic sheet 102, the second metallic sheet 104, and the support structure 100 and each bolt 106 (used to attach the layers of the hardening filter 96 to the carriage 56) may pass through each of the concentric holes 108 present in each layer. As an example, one end of the hardening filter 96 may be attached to the tab 90 of the carriage 56. In one example, the support structure 100 may be made of a metal such as aluminum, and first metallic sheet 102 and the second metallic sheet 104 may be made of a same metal or different metals. Copper may be used to form one or both of the first metallic sheet 102 and the second metallic sheet 104.

The hardening filter 96 may be used to intercept lower energy radiation, thereby attenuating low energy radiation and “hardening” the x-ray beam 16 passing through the hardening filter 96. The degree of beam attenuation may depend on one or more of a number of attenuation layers (such as metallic sheets), the thickness of each attenuation layer, the materials used in the attenuation layers, and the overall size of the attenuation layers. The degree of beam attenuation is much higher with heavy elements. Heavy elements, like tungsten, absorb incoming x-rays and emits their unique K-xray that is monoenergetic. A Monoenergetic beam is mainly intended for detector energy threshold calibration in clinical application due to its limited intensity. In preclinical CT, it can be used for small animal imaging as well.

As an example, when using thinner or weaker sheets of hardening material in 96, the support plate 100 may be used to limit deflection due to gantry rotational forces which may act to bend the middle of the hardening material. In this embodiment, the support plate 100 is positioned outside of the cross-sectional area of the hardened x-ray beam 16 that is used for imaging. In this way, the hardening filter 96 may be solely accounted for in hardening the imaging x-ray beam 16 while being mechanically strengthened by the support plate 100 proximal to the area where the imaging beam 16 passes through the hardening filter 96. Furthermore, the support plate 100 may be made from a stiff but lightweight material such as aluminum to minimize excess x-ray scatter near the hardening filter.

An x-ray beam filter 110 may be coupled to the underside of the carriage 56 and may extend along the entire lower surface of the carriage 56. The filter may further condition the x-ray beam 16 after the beam has passed through one or more of the hardening filter and bowtie filters.

During an imaging, an x-ray beam 16 may first pass through the hardening filter 64 followed by a bowtie filter (e.g., a bowtie filter 60 of the second filter assembly 52, depicted in FIG. 6). The carriages 54, 56 may be moved along a direction perpendicular to that of the beam 16 to position the beam on a bowtie filter 60 and/or the hardening filter 64. A level of beam attenuation and spatial distribution may be adjusted by selecting a hardening filter 64 and/or bowtie filters 58, 60, 62. In one example, the carriages 54, 56 may be positioned such that the beam passes through the hardening filter 64 and the second bowtie filter 60, the hardening filter 64 overlapping with the second bowtie filter 60. In another example, the carriages 54, 56 may be positioned such that the beam passes the first bowtie filter 58 only, the second bowtie filter 60 only, or the third bowtie filter 62 only. In yet another example, the carriages 54, 56 may be positioned such that the beam passes through the hardening filter 64 only. After passing through one of the second or third bowtie filters 60, 62, the beam also passes through the aluminum filter 110 before entering a subject that is scanned. In some examples, the second carriage 56 may be positioned such that the beam passes through the hardening filter 96 on the second carriage 56 and one of the second or third bowtie filters 60, 62. In some examples, the second carriage 56 may be positioned such that the beam passes through the hardening filter 96 on the second carriage 56 only.

Attenuation of the beam via a hardening filter may be specifically desired during a scout scan which may precede a diagnostic scan. During a diagnostic scan, a bowtie filter without the hardening filter may be used for diagnostic scans. Typically, for a scout scan a smaller beam (coverage) may be used relative to the beam size used for diagnostic scans. The smaller beam may completely pass through the hardening filter 96 which is narrower than a bowtie filter. Also, by using a hardening filter 64, 96, a higher power x-ray source with increased x-ray tube temperature may be used during a scan without increasing radiation exposure of the subject. The higher power may improve the quality of the scout scan and/or subsequent diagnostic scans and improve thermal stability of the x-ray tube including the target. The consistently higher temperature of the x-ray tube target may contribute to long-term reliability of the device as it remains closer to an optimal operating temperature; fewer temperature cycles of the internal parts contribute to better reliability.

FIGS. 7A and 7B depict cross-sectional views of the first example integrated filter assembly 50 of FIGS. 3-5 and the second example integrated filter assembly 52 of FIG. 6 positioned within the example imaging system 10 of FIG. 1. As depicted in FIGS. 7A and 7B, the filter assemblies 50, 52 may be positioned such that the first hardening filter 64 of the first filter assembly 50 is positioned in the path of the beam 16. In this position, the hardening filter 64 is the only filter positioned in the path of the beam 16. This position of the filters may be preferred for certain imaging operations, such as calibration of the imaging system. In another example position of the first and second filter assemblies 50, 52, the hardening filter 64 of the first assembly 50 and a bowtie filter of the second assembly 52 (e.g., the second bowtie filter 60) may both be positioned within the path of the beam 16. In this illustrated position, the hardening filter 64 overlaps the second bowtie filter 60.

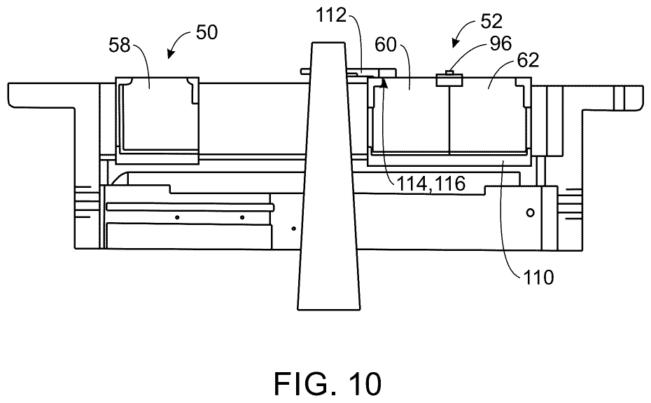

FIGS. 8-10 depict cross-sectional views of alternative configurations of the example integrated filter assembly 50 of FIGS. 3-5 and the example integrated filter assembly 52 of FIG. 6 positioned within the example imaging system 10 of FIG. 1. In the example configuration illustrated in FIG. 8, the first filter assembly 50 is the same as the example first filter assembly 50 described in FIGS. 3-5. However, the second filter assembly 52 does not include the hardening filter 96 positioned between the second and third bowtie filters 60, 62. FIG. 9 depicts an alternate configuration in which the first filter assembly 50 does not include any hardening filters, and the second filter assembly 52 includes a hardening filter 112 coupled to an edge 114 of a top surface 116 of the second carriage 56. The example hardening filter 112 may be similar to the hardening filter 64 described in conjunction with FIGS. 3-5. In FIG. 10, the depicted alternative configuration includes a first filter assembly 50 that does not include any hardening filters, and a second filter assembly 52 that includes a hardening filter 112 coupled to the edge 114 of the top surface 116 of the carriage 56 and the hardening filter 96 positioned between the second and third bowtie filters 60, 62. While three alternative configurations are depicted in FIG. 8-10, any number of configurations of filter assemblies may be used with a hardening filter (e.g., hardening filter 64, 114) coupled to an edge of a top surface of a carriage, as described herein.

FIGS. 11A-11F show a variety of example positions of the filter assemblies 50, 52. In particular, FIGS. 11A-11F depict the three bowtie filters 58, 60, 62 in their respective carriages 54, 56 and the first hardening filter coupled 64 to the first filter assembly 50. As an example, each of the three filters 58, 60, 62 may be bowtie filters. In this example, the first bowtie filter 50 and the hardening filter 64 are positioned together in the first filter assembly carriage 54, and the second and third bowtie filters 60, 62 are positioned within the second filter assembly carriage 56.

The first carriage 54 may be coupled to a first shaft 120, and the carriage 54 may be translated along a first shaft 120 by rotating the first shaft 120 with a first motor 122. The first shaft 120 may be a screw, ballscrew, or a similar design to translate rotational motion into linear motion of the first carriage 54. The second carriage 56 may be coupled to a second shaft 126 and may be translated along a second shaft 126 by rotating the second shaft 126 with a second motor 128. The second shaft 126 may be a screw, ballscrew, or a similar design to translate rotational motion into linear motion of the second carriage 56. A localized clearance feature (not shown) is present in the second carriage 56 to avert interference of the first shaft 120 with the second carriage 56 as the second carriage 56 translates along the second shaft 126. The center of the x-ray beam 16 (such as x-ray radiation 16 of FIGS. 1-2) is indicated by a horizontal line. One of the three filters 58, 60, 62 along with the hardening filter 64 may be selectively translated into the beam path of the x-ray beam 16 by rotating one or both shafts 120, 126 via motors 122, 128, respectively. The first and the second shafts 120, 126 may be aligned in one line, and are spaced apart from each other by a gap. The x-ray beam 16 may transmit through the gap. The motor (such as motor 122, 128), the shaft (such as shaft 120, 126) coupled to the motor 122, 128, and the filter assembly (such as filter assembly 50, 52) coupled to the shaft 120, 126 may form a filter driving system 130, 132. The filter assembly 50, 52 may include one or more filter driving systems 130, 132.

FIG. 11A shows a first position 134 of the filter assemblies 50, 52. The x-ray beam 16 transmits through the filter housing 136 without passing through any filter. The first carriage 54 may be located closer to the first motor 122, and the second carriage 56 may be located closer to the second motor 128.

FIG. 11B shows a second position 138 of the filter assemblies 50, 52. The x-ray beam 16 transmits though the third filter 62 in the second carriage 56. The second filter assembly 52 may transit from the first position 134 to the second position 138 by actuating the second motor 128 and translating the third filter 62 (in carriage 56) into the x-ray beam path 16.

FIG. 11C shows a third position 140 of the filter assemblies 50, 52. The x-ray beam 16 solely transmits though the second filter 60 in the second carriage 56. The second carriage 56 may transit from the first position 134 or the second position 138 to the third position 140 by actuating the second motor 128 and translating the second filter 60 into the x-ray beam path 16.

FIG. 11D shows a fourth position 142 of the filter assemblies 50, 52. The x-ray beam 16 transmits through the hardening filter 64. The filter assembly 50 may transit from any of the above-mentioned first 134, second 138, or third position 140 to the fourth position 142 by actuating the first motor 122 to translate the first carriage 54 further from the first motor 122, and subsequently or simultaneously actuating the second motor 128 to translate the second carriage 56 out of the x-ray beam path 16, as needed.

FIG. 11E shows a fifth position 144 of the filter assemblies 50, 52. The x-ray beam 16 transmits through the hardening filter 64 and the second filter 60. The filter assemblies 50, 52 may transit from any of the above-mentioned first 134, second 138, third 140, or fourth 142 position to the fifth position 144 by actuating the first motor 122 to translate the first carriage 54 relative to the first motor 122, as needed, and subsequently or simultaneously actuating the second motor 128 to translate the second filter 60 in the second carriage 56 into the x-ray beam path 16, as needed.

FIG. 11F shows a sixth position 146 of the filter assemblies 50, 52. The x-ray beam 16 transmits through the first filter 58. The filter assembly 50 may transit from any of the above-mentioned first 134, second 138, third 140, fourth 142, and fifth 144 position to the sixth position 146 by actuating the first motor 122 to translate the first carriage 54 further from the first motor 122, and subsequently or simultaneously actuating the second motor 128 to translate the second carriage 56 out of the x-ray beam path 16, as needed.

Based on the instructions stored in the non-transient memory, the computing device (such as computer 36 of FIG. 2) may move the filter assemblies 50, 52 from any one of the above positions to another position by actuating one or more of the two motors 122, 128. In one embodiment, one filter and a hardening filter are positioned in one carriage and two filters are positioned in the other carriage. As one example, the two filters may be coupled to one shaft and driven by one motor. In another embodiment, more than three filters and multiple hardening filters may be arranged within the filter housing 136. For example, the numbers of filters coupled to each shaft are the same, if the total number of filters in the housing is even. The numbers of filters coupled to each shaft is different, if the total number of filters in the housing is odd.

In yet another embodiment, the arrangement of the filters in the filter housing 136 may be based on the type of the filters. Herein, the filter type may be determined by the section of the subject that the filter is designed to image. For example, the first filter 58 used for imaging the first section of the subject 22 and the second filter 60 used or imaging the second section of the subject 22 may be positioned next to each other, if the first section and the second section are connected. The first filter 58 and the second filter 60 may be positioned apart from each other (such as separated by another filter), if the first section and the second section are not connected. As an example, the filter for imaging the abdomen maybe positioned next to the filter for imaging the chest, but apart from the filter for imaging the head. In this way, when the chest is imaged after imaging the abdomen, the switching of filters is simpler to achieve with less overall carriage motion. The hardening filter may be coupled between two filters which may be used for a scout scan. Locating the hardening filter in this location makes the switching from scout scan to diagnostic scanning simpler with less overall carriage motion.

In other embodiments, a carriage including filters may be translated with any one of a rack and pinion, a belt, or a cable-driven system in lieu of a shaft.

FIGS. 12A-12C depicts scans using the example filter assembly in various positions described in relation to FIGS. 11A-11F. For example, FIG. 12A depicts an example scan using the imaging system 10 where the filter assemblies 50, 52 are arranged such that only a bowtie filter is in the path of the beam 16. For example, FIG. 12A depicts the filter assemblies 50, 52 in one of the second 138, third 140 or sixth 146 positions, where only one of the bowtie filters 58, 60, 62 is in the path of the beam 16. FIG. 12B depicts an example scan using the imaging system 10 where the filter assemblies 50, 52 are arranged such that a bowtie filter and a hardening filter are in the path of the beam 16. For example, FIG. 12B depicts the filter assemblies 50, 52 in the fifth position 144, where the second bowtie filter 60 and the hardening filter 64 are in the path of the beam 16. FIG. 12C depicts an example scan using the imaging system 10 where the filter assemblies 50, 52 are arranged such that only a hardening filter is in the path of the beam 16. For example, FIG. 12C depicts the filter assemblies 50, 52 in the fourth position 142, where the hardening filter 64 is in the path of the beam 16.

FIGS. 13-16 depict additional example filter assemblies that may be used in the pre-patient collimator assembly in place of the example filter assemblies depicted in FIGS. 3-5. The filter assembly 148 may include a carriage 150 including at least one bowtie filter. In particular, the example filter assembly 148 depicted in FIG. 13 includes one first bowtie filter 152 positioned within the carriage 150. Additionally, the example carriage 150 may include one or more hardening filter 154, 156. In some examples, the carriage may include a third hardening filter 158 extending from the example hardening filter 156, as shown in FIG. 16. FIG. 16 depicts an example filter assembly 149 including an example carriage 151 that is substantially similar to the carriage 150 of FIG. 13, but includes an additional hardening filter 158. In the illustrated example of FIG. 13, the first carriage 150 includes a first hardening filter 154 and a second hardening filter 156 coupled to an extending from a top edge 160 of the carriage 150. In the illustrated example, the first and second hardening filters are positioned in individual corresponding windows 162, 164 of a frame member 168. As depicted in FIG. 16, the third hardening filter 158 is positioned in a corresponding third window 166 of the frame member 168. Alternatively, the plurality of hardening filters 154, 156, 158 may be positioned within a single window (i.e., the window is larger to accommodate the plurality of hardening filters being positioned side-by-side). The frame member 168 is depicted as being coupled to the top edge 160 by a plurality of fasteners, such as bolts or screws. Alternatively, other types of fasteners may be used, including, but not limited to, adhesives, brackets, rivets, or clips. In some examples, the frame member 168 is integrally designed with the carriage.

The example hardening filters 154, 156, 158 may be secured in the frame member 168 using an additional bracket (similar to that depicted in FIGS. 3-5) positioned on the underside of the frame member 168 such that each filter is sandwiched between the frame member 168 and the additional bracket. In such examples, the hardening filters 154, 156, 158 may be secured within the frame member 168 via a plurality of fasteners (e.g., bolts, screws, clips, etc.). Alternatively, the example frame member 168 may include grooves or slots through which each of the hardening filters may be moved to position each hardening filter 154, 156, 158 within the frame member 168.

The example hardening filters 154, 156, 158 made of different materials and/or may have different thicknesses. For example, one of the example hardening filters may be made of a first material and one of the other hardening filters may be made of a second material. In a specific example, one hardening filter may be made of tungsten or tungsten alloy while the other hardening filter(s) made of copper or copper alloy. For example, in the illustrated example of FIG. 16, which includes three hardening filters 154, 156, 158, the hardening filter closest to the carriage 151 may be made of the heavier material (e.g., tungsten compared to copper used for the other hardening filters 156, 158). In other examples, such as the example illustrated in FIG. 13, each of the hardening filters 154, 156 is made of the same material. Additionally or alternatively, example carriages having two hardening filters may include filters of different materials, example carriages having three hardening filters may include filters of the same material, and/or example carriages having three hardening filters may include filters of three different materials.

Similarly, the hardening filters may be different thicknesses. For example, different materials may require different thicknesses, or different thicknesses of hardening filters of the same materials may be used for different purposes. For example, in embodiments where two of the filters are made of one material (e.g., copper or copper alloy), one of the hardening filters may have a thickness of approximately 0.5 mm and the other of the hardening filters may have a thickness of approximately 1.55 mm. The thicker hardening filter may be more suitable for scout and/or calibration scans, while thinner hardening filter is better for diagnostic scans because the thinner filter is more likely to be paired with a bowtie filter during a diagnostic scan. In examples where the carriage includes three hardening filters, the third filter may have yet another thickness. For example, the third filter may have a thickness less than the thickness of the other two hardening filters. In one particular example, the first hardening filter may be made of tungsten with a thickness of 00.4 mm, the second hardening filter may be made of copper with a thickness of 1.5 mm, and the third hardening filter may be made of copper, with a thickness of 1.55 mm. In other examples, the third filter may have a thickness greater the thickness of the other two filters (e.g., a thickness greater than 1.5 mm, in one particular example). While the hardening filters may be positioned in any order, it may be advantageous to position the heaviest filter (e.g., a filter made of a heavier material, such as tungsten, or a thicker filter) closest to the carriage.

A certain one of the hardening filters may be more suited to a particular scanning operation. For example, one of the example filters may be more suited for a calibration scan, one of the example filters may be more suited for a scout scan, and one of the example filters may be more suited for a diagnostic scan. However, any of the example hardening filters can be used for each type of scan (e.g., a calibration scan, a scout scan, a diagnostic scan) and the filter used may be selected by the technologist based on patient characteristics (e.g., size, age, etc.) and/or scan parameters (e.g., region of interest, type of scan, desired field of view etc.). Additionally, a certain material or thickness of hardening filter may be selected if a goal is dose reduction (or improving dose efficiency) during the scan. While tungsten and copper are mentioned as example materials, other materials that may be used include silver, tin, ytterbium, hafnium, aluminum, and bismuth. Additionally, any combination or alloy of these materials (as well as copper and tungsten) may be used. Furthermore, while example thicknesses (0.4 mm, 0.5 mm and 1.55 mm) were given the thickness of the example hardening filters may be varied based on material used and/or desired dose efficiency. To decide on the number, thickness and materials of the example hardening filters, factors such as available space, image quality, dose reduction/dose efficiency, consistency, and material availability were evaluated.

In the illustrated example, the widths of the hardening filter are approximately equal. However, in some examples, the widths may be different. For example, a narrower hardening filter may be used for a smaller scan range or detector size, while a wider hardening filter may be used for a larger scan range or detector size. In the illustrated example, the hardening filters having a size corresponding to an 80 mm beam width, which is approximately half of the scan range of a typical bowtie filter (such as those depicted in FIGS. 3-6). In other examples, the hardening filters may have a width corresponding to a 160 mm beam width, or other size of scan range.

The example carriage 150 also includes a single bowtie filter 152. In other examples, multiple bowtie filters may be positioned within the carriage 150. In some examples, the bowtie filter 152 may have a different width than the hardening filters. For example, the width of the bowtie filter 152 may correspond to a 160 mm beam width. A bowtie filter with a width corresponding to a 160 mm beam width may be referred to herein as a medium bowtie filter or standard width bowtie filter, but it should be understood that the terms “medium” and “standard” may vary based on application and/or product line, and in other applications a medium or standard bowtie filter may correspond to a different beam width and/or may correspond to two different beam widths. In examples where three (or more) beam hardening filters 154, 156 are coupled to the top edge 160, a smaller bowtie filter (e.g., a small bowtie filter, a bowtie filter having a width corresponding to less than a 160 mm beam width) may be positioned within the carriage 150 to allow for additional space for the additional hardening filter. In some examples, a small bowtie filter may be approximately half the width of the medium bowtie filter and/or have a width corresponding to an 80 mm beam width. However, any width smaller than the width of the medium filter may be sufficient.

FIG. 14 depict another example filter assembly 169 including an example carriage 170 that may be used in conjunction with the carriage 150 of FIG. 13. The carriage 170 is substantially similar to the carriage of 56 of FIG. 6. The example carriage 170 may include two or more bowtie filters 172, 174. While not depicted, in some examples, an additional beam hardening filter may be positioned across the top of the carriage 170 and between the two filters 172, 174, as described in conjunction with the carriage 56 of FIG. 6.

The example two bowtie filters 172, 174 are depicted as being equal in width. In such examples, both bowtie filters may be medium width bowtie filters. Alternatively, the bowtie filters 172, 174 may be different widths corresponding to different beam widths. For example, one filter may be a small width filter and the other may be a medium width filter. Alternatively, one filter may be a small width filter and the other may be a large width filter. A large width filter may have a width corresponding to a beam width of 240 mm. In such examples, the small width filter and the large width filter fill the same space as two medium width filters. Alternatively, any filter having a width greater than the medium width filter may be considered a large width filter. In some examples, the filters are positioned within the carriages 150, 170 such that, between the two carriages, there is a small width filter, a medium width filter, and a large width filter.

Additionally, the bowtie filters 152, 172, 174 are shown here in rectangular shape as an example. The bowtie filters 152, 172, 174 may alternatively have different shapes and material constructions to provide proper x-ray special spectrum for imaging various types of anatomics. The bowtie filter(s) 152, 172, 174 may change the spatial distribution of the radiation beam (i.e., condition the beam) in the axial plane of the imaging subject 22 (such as a patient). For example, the re-distributed radiation beam 16 may have higher energy at the center and lower energy at the periphery of the subject 22. Each of the bowtie filters 152, 172, 174 may be designed to image a specific anatomy or section of the human body, such as head, chest, and abdomen. Thus, a smaller bowtie filter may be more suitable to scan, for example, a head of the subject 22. During imaging, one of the bowtie filters 152, 172, 174 may be selected based on the anatomy of the subject 22 to be scanned, and the selected filter may be placed into the radiation beam path 16. Responsive to a change in the anatomy, the filter(s) may be changed from one to another. Based on a nature of the scan, the carriage(s) may be positioned such that a hardening filter may or may not be placed in the radiation beam path 16. The hardening filter(s) may attenuate the beam 16 and remove low energy components thereby conditioning the beam 16 for specific scans, such as a patient scan or a calibration.

Similarly to the example carriages 54, 56 of FIGS. 3-6, the carriages 150, 170 may be moved relative to each other to enable various combinations of filters being placed such that the beam 16 passes through the filters prior to passing through the subject 22. FIGS. 15 and 17A-F depict some example different configurations or options for positioning the carriages 150, 170 relative to the beam 16. It should be understood that, while all of the filters are depicted as equivalent widths for simplicity, the filters may have different widths in accordance with the discussion of FIGS. 13 and 14 above.

FIG. 15 depicts an example perspective view of the carriages 150, 170 positioned adjacent each other, such that a beam 16 may pass through both a hardening filter 156 and a carriage 174 prior to passing through the subject 22. While the example of FIGS. 15 and 17A-F depict the carriage 150 of FIG. 13 positioned adjacent to the carriage 170 of FIG. 14, another carriage (e.g., the carriage 151 of FIG. 16, the carriage 50 of FIGS. 3-5) may be positioned adjacent to the carriage 170 instead. A beam 16 may also pass through hardening filter 154 and a carriage 174 prior to passing through the subject 22. As depicted in FIG. 14, the hardening filters 154, 156 are coupled to the carriage 150 such that the hardening filters 154, 156 may pass over or on top of the bowtie filters 172, 174 of the other carriage 170. This configuration prevents collision between the frame 168 and the carriage 170. In other embodiments, it is possible that the carriages may be design such that the hardening filters 154, 156 and the frame 168 are coupled to a bottom surface of the carriage 150, such that the hardening filters 154, 156 are positioned below or under the carriage 170.

FIGS. 17A-17F schematically depict multiple positions of the example carriages 150 and 170 relative to each other and the beam 16. It can be understood from these figures that there may be additional combinations made possible by including additional hardening filters (e.g., a third hardening filter), changing the width of the hardening filters, and/or changing the width of the bowtie filters. It is also understood that these figures merely represent some example configurations or positions, and there are other configurations that are not depicted but that may be within the scope of this invention. The example carriages 150, 170 may be driven or controlled in the same manner as the carriages 54, 56 described in conjunction with FIGS. 11A-F, and similar parts will be given similar part numbers.

FIG. 17A shows a first position 176 of the carriages 150, 170. The x-ray beam 16 transmits through the filter housing 136 without passing through any filter. The first carriage 150 may be located closer to the first motor 122, and the second carriage 170 may be located closer to the second motor 128.

FIG. 17B shows a second position 178 of the carriages 150, 170. The x-ray beam 16 transmits though the second beam hardening filter 156 attached to the carriage 150. The carriage 150 may transit from the first position 176 to the second position 178 by actuating the first motor 120 and translating the carriage 150 until the second hardening filter 156 is in the x-ray beam path 16.

FIG. 17C shows a third position 180 of the carriages 150, 170. The x-ray beam 16 solely transmits though the second hardening filter 156 and the second bowtie filter 174 of the carriage 170. The second carriage 170 may transit from the first position 134 to the third position 180 by actuating the second motor 128 and translating the carriage 170 until the bowtie filter 174 is in the x-ray beam path 16. In some examples, the carriage 150 is also moved from the first position 176 to the third position 180. In the illustrated example, the second and third positions for the carriage 150 may be substantially the same.

FIG. 17D shows a fourth position 182 of the carriages 150, 170. The x-ray beam 16 transmits through the first hardening filter 154 and the second bowtie filter 174 of the carriage 170. The carriage 150 may transit from any of the above-mentioned first 176, second 176, or third position 180 to the fourth position 182 by actuating the first motor 122 to translate the first carriage 150 further from the first motor 122, and subsequently or simultaneously actuating the second motor 128 to translate the second carriage 170, as needed. Additionally or alternatively, in some instances, the carriages 150, 170 may be moved such that the first bowtie filter 172 of the carriage 170 and the second beam hardening filter 156 are positioned within the x-ray beam path, though this position is not depicted.

FIG. 17E shows a fifth position 184 of the carriages 150, 170. The x-ray beam 16 transmits through just the bowtie filter 150 of the carriage 150. The carriages 150, 170 may transit from any of the above-mentioned first 176, second 178, third 180, fourth 182, or any other conceivable position, to the fifth position 184 by actuating the first motor 122 to translate the first carriage 150 relative to the first motor 122, as needed, and subsequently or simultaneously actuating the second motor 128 to translate the second carriage 170 into or out of the x-ray beam path 16, as needed.

FIG. 17F shows a sixth position 186 of the filter carriages 150, 170. The x-ray beam 16 transmits through just the second bowtie filter 174 of the carriage 170. It should also be understood that the carriage 170 may be adjusted such that the x-ray beam transmits though just the first bowtie filter 172, though this position is not depicted. The carriage 170 may transit from any of the above-mentioned first 176, second 178, third 180, fourth 182, fifth 184, or any other conceivable position, to the sixth position 186 by actuating the first motor 122 to translate the first carriage 150 relative to the first motor 122, and subsequently or simultaneously actuating the second motor 128 to translate the second carriage 170 into or out of the x-ray beam path 16, as needed.

In this way, FIGS. 1-17F provide for an imaging system, comprising a gantry for receiving an imaging subject, a radiation source positioned in the gantry for emitting radiation exposure, a detector positioned on the opposite of the gantry relative to the radiation source, a motorized table for moving the imaging subject within the gantry, a computation device with instructions stored in a non-transient memory, filter carriages mounted to the gantry, one or more bowtie filters positioned within one of the filter carriages, and at least one hardening filter positioned in one of the filter carriages, and a filter driving system for switching filters by moving one of the bowtie filters and/or one of the hardening filters into or out of the radiation beam.

FIGS. 18 and 19 are graphical representations of energy spectrum curves that depict the effect of the filters 64 of FIG. 3 depending on the material, tungsten and Al/Cu, respectively. Using the tungsten hardening filter results in a monoenergetic x-ray beam of the specific energy (e.g., 69.5 kv, FIG. 18). The monoenergetic beam is used for the energy calibration of clinical photon counting CT detector. It can also be used for preclinical small animal imaging to achieve better image quality since a mono-energy beam, like a synchrotron x-ray radiation source, is an ideal x-ray source for various imaging applications if high beam intensity can be achieved. Al/Cu filter absorbs low energy x-rays and passes through high energy x-rays, FIG. 19, that are used for patient imaging. Since it is increasing the average energy of the beam, it is called a beam hardening filter. Those low energy x-rays are mostly expected to be absorbed in patients as well and so, not contributing patient image quality but increasing patient dose. FIG. 18 and FIG. 19 show different filter characteristics, but they both are called a hardening filter in this document.

FIG. 20 shows an example method 200 for performing calibration scans using hardening filter(s) in an integrated filter assembly (such as integrated filter assemblies 50, 52 or carriages 150, 170 in described here). Method 200 achieves calibration of an imaging system (such as imaging system 10) by using a hardening filter (such as hardening filters 64, 154, 156) that is positioned within a filter assembly. Additionally, a phantom may be used during the calibration, or the calibration may be performed as an air scan. Method 200 and all methods described herein may be performed according to instructions stored in the non-transitory memory in a computing device (such as computer 36 of FIG. 2) of the imaging system 10.

At 202, a subject (such as subject 22 in FIG. 2, a phantom, or no subject) of the calibration scans may be positioned on a motorized table (such as table 46 in FIG. 1). A table motor controller 44 may move the table 46 so that a proper section of the subject 22 is within the gantry 12 for imaging.

At 204, the routine includes determining if a calibration scan is desired. A calibration scan may be used to calibrate one or more components of the imaging system, including x-ray detector, x-ray tube, x-ray dose, gantry weight balance, and/or software or firmware used to process data collected by the imaging system. Calibration scans may also be used to detect misalignment in the imaging system.

If it is determined that a calibration scan is desired, at 206, scan parameters may be set up for carrying out a calibration scan. For example, a user may input or select the scan parameters according to a scanning protocol or a menu. The scan parameters may include the type and sequence of the filters that are going to be used during the scan. As an example, for a calibration scan a hardening filter may be used for conditioning the x-ray beam used for imaging the subject. Scan parameters may also include setting scan timing. As one example, the scan timing may include a start time and a duration for imaging each section.

At 208, a hardening filter may be positioned in the path of the x-ray beam by operating a motor coupled to a carriage including the hardening filter (such as hardening filter 64 in FIG. 3 or hardening filters 154, 156, 158 of FIG. 13 and FIG. 16). The carriage may be moved along a shaft in a plane perpendicular to the plane of the x-ray beam to position the hardening filter in the beam. The controller may actuate the motor to move the shaft and the carriage to the desired position. A hardening filter may intercept lower energy radiations, thereby attenuating and “hardening” the beam.

At 210, method 200 may start acquiring the dataset of the calibration scan. For example, the radiation source (such as 14 of FIGS. 1-2) may be activated, and radiation exposure of the imaging subject through the hardening filter may be started.

The dataset is acquired from the detector (such as 18 of FIG. 2) upon receiving the transmitted radiation signal from the imaging subject. As one example, the anatomy or positioning of the imaging subject may be monitored by analyzing the acquired dataset. The currently imaged location may be calculated based on the starting location of the scan and the travel distance of the motorized table. In one embodiment, the anatomies or positions of the subject may be grouped in different types. As another example, the collected data can be analyzed, for example, K-edge search to locate 69.5 keV in electronics, and produce its result. The result can be used to decide to finish or repeat the calibration.

At 212, the routine includes determining if the calibration scan has ended. The end of the calibration scan may be determined based on the protocol setup at step 206. If it is determined that the calibration scan has not ended, at 214, the scout scan may be continued, and data may be acquired. If it is determined that the calibration scan has ended, the calibration is complete.

FIG. 21 shows an example method 300 for performing image scans using multiple filters included in an integrated filter assembly (such as integrated filter assembly 50, 52, carriages 150, 151, 170). Method 300 achieves image acquisition of the imaging subject by changing filters between successive scans. Method 300 and all methods described herein may be performed according to instructions stored in the non-transitory memory in a computing device (such as computer 36 of FIG. 2) of the imaging system.

At 302, a subject (such as subject 22 in FIG. 2) of the imaging scans may be positioned on a motorized table (such as table 46 in FIG. 2). A table motor controller 44 may move the table so that a proper section of the subject is within the gantry for imaging.

At 304, the routine includes determining if a scout scan is desired. A scout scan provides a projection view along a longitudinal axis of the imaging subject and generally provides aggregations each including internal structures of the subject. During a scout scan, while all the components of the imaging system may be maintained in a stationary position, the subject may be passed through the imaging system to perform a scan on the subject. A scout scan may be used to identify a region of interest of the subject for a subsequent diagnostic scan.

If it is determined that a scout scan is desired, at 306, scan parameters may be set up for carrying out a scout scan. For example, a user may input or select the scan parameters according to a scanning protocol or a menu. The scan parameters may include the type and sequence of the filters that are going to be used during the scan. As an example, for a scout scan a bowtie filter along with a hardening filter may be used for conditioning the x-ray beam used for imaging the subject. Scan parameters may also include setting scan timing. As one example, the scan timing may include a start time and a duration for imaging each section.

At 308, a bowtie filter and a hardening filter may be positioned in the path of the x-ray beam by operating a motor(s) coupled to a carriage(s) including the bowtie filter (such as bowtie filter 60 in FIG. 6, or bowtie filters 152, 172, 174 of FIGS. 13 and 14) and the hardening filter (such as hardening filter 64 in FIG. 3 or hardening filters 154, 156, 158 of FIGS. 13 and 16). The carriage(s) may be moved along a shaft in a plane perpendicular to the plane of the x-ray beam to position the bowtie filter and the hardening filter in the beam. The controller may actuate the motor to move the shaft and the carriage to the desired position. The bowtie filter may change the spatial distribution of the radiation beam in the axial plane of the imaging subject (such as a patient). For example, the re-distributed radiation beam may have higher energy at the center and lower energy at the periphery of the subject. A hardening filter may intercept lower energy radiations, thereby attenuating and “hardening” the beam. The hardening filter may at least partially overlap with the bowtie filter and the beam may first pass through the hardening filter and then enter the bowtie filter.

At 310, method 300 may start acquiring the dataset of the imaging subject. For example, the radiation source (such as 14 of FIGS. 1-2) may be activated, and radiation exposure of the imaging subject through the bowtie filter and the hardening filter may be started. For a scout scan, a smallest permissible beam may be used. In one example, the beam may be 5 mm. By using a hardening filter to attenuate the beam reaching the subject, a higher power x-ray source with increased x-ray tube temperature may be used during the scout scan without increasing radiation exposure of the subject. The higher power improves the quality of the diagnostic scan and improves thermal stability of the x-ray tube including the target. In one example, a 50 kW x-ray power scan technique (100 kV, 500 mA) may be used.

The dataset is acquired from the detector (such as 18 of FIG. 2) upon receiving the transmitted radiation signal from the imaging subject. As one example, the anatomy of the imaging subject may be monitored by analyzing the acquired dataset. As another example, the anatomy of the imaging subject may be estimated by the currently imaged location. The currently imaged location may be calculated based on the starting location of the scan and the travel distance of the motorized table. In one embodiment, the anatomies of the subject may be grouped in different types. For example, the anatomy of a human body may be grouped based on size, type such as the head, the chest, and the abdomen.

At 312, the routine includes determining if the scout scan has ended. The end of the scout scan may be determined based on the protocol setup at step 306. If it is determined that the scout scan has not ended, at 314, the scout scan may be continued, and data may be acquired.

If it is determined that the scout scan has ended, at 316, the routine includes determining if a diagnostic scan is desired. As an example, a decision to carry out the diagnostic scan may be made based on the images reconstructed from the data acquired during the scout scan. The image from the scout scan may be two-dimensional or three-dimensional. Based on the scout scan, a specific anatomy may be selected for a diagnostic scan. The diagnostic scan may provide a detailed image of the specific anatomy which might not be available via the scout scan.

If at 304, it is determined that a scout scan is not desired, the routine may directly proceed to step 316 for determining if a diagnostic scan is desired. A scout scan may not always precede a diagnostic scan.

If it is determined that a diagnostic scan is not desired and a scout scan has been completed, at 318, the acquired dataset from the scout scan is displayed and stored. In one embodiment, dataset acquired from different sections of the subject may be re-constructed to form an image. The acquired dataset, as well as the processed images may be saved in the storage of the imaging system and no further scans may be carried out. The routine may then end.

If it is determined that a diagnostic scan is desired, the routine may proceed to step 320 wherein the scan parameters may be set up for carrying out a diagnostic scan. A user may input or select the scan parameters according to a scanning protocol or a menu. The scan parameters may include the type and sequence of the filters that are going to be used during the scan. The type of the filters may be chosen based on the anatomy of imaging subject that is to be imaged. The parameters may also include setting scan timing. As one example, the scan timing may include a start time and a duration for imaging each section. Anatomy information of the imaging subject may be loaded to the memory of the computation device. The anatomy information may be acquired from a pre-scan. The anatomy information may be acquired from the prior scout scan or a localized scan. This step may also include moving the imaging subject via the motorized table so that the proper section of the subject is within the gantry for imaging.

At 322, a contrast agent may be injected into the imaging subject. The contrast agent may enhance the contrast of images captured specifically for certain anatomies. This step is optional and the diagnostic scan may be carried out without use of a contrast agent.

At 324, a bowtie filter may be positioned in the path of the x-ray beam by operating a motor coupled to a carriage including the bowtie filter. The type of the filter may be determined based on the anatomy of the currently imaged section of the subject. The carriages may be moved along a shaft in a plane perpendicular to the plane of the x-ray beam to position the bowtie filter in the beam. In one example, the bowtie filter used for the diagnostic scan may be same as the bowtie filter used in the scout scan. In another example, the bowtie filter used for the diagnostic scan may be different from the bowtie filter used in the scout scan. In this way, a single carriage including one or more bowtie filters and/or a hardening filter may be used for both the scout scan and the diagnostic scan without the need for additional components. Furthermore, multiple filter carriages can be used together to position a bowtie filter with or without a hardening filter into the x-ray beam.

At 326, dataset of the imaging subject may be acquired. For example, the radiation source may be activated, and radiation exposure of the imaging subject through the selected bowtie filter may be started. For a diagnostic scan, a beam size of 25 mm-160 mm at the filter assembly may be used. The dataset is acquired from the detector upon receiving the transmitted radiation signal from the imaging subject. As one example, the anatomy of the imaging subject may be monitored by analyzing the acquired dataset. As another example, the anatomy of the imaging subject may be estimated by the currently imaged location. The currently imaged location may be calculated based on the starting location of the scan and the travel distance of the motorized table. In one embodiment, the anatomies of the subject may be grouped in different types. For example, the anatomy of a human body may be grouped based on size, types of such as the head, the chest, and the abdomen.

At 328, the routine includes determining if the diagnostic scan has ended. The end of the diagnostic scan may be determined based on the protocol setup at step 320. If it is determined that the diagnostic scan has not ended, a 330, the diagnostic scan may be continued and data may be acquired.

If it is determined that the diagnostic scan has ended, the acquired dataset from the diagnostic scan is displayed and stored. In one embodiment, dataset acquired from different sections of the subject may be re-constructed to form an image. The acquired dataset, as well as the processed images may be saved in the storage of the imaging system and no further scans may be carried out. The routine may then end.

In this way, during a first imaging (such as a scout scan), carriages may be moved to position a hardening filter and a first bowtie filter housed in the carriages in a path of a radiation beam between a radiation source and an imaging subject and during a second imaging (such as a diagnostic scan), the carriages may be moved to move the hardening filter and the first bowtie filter out of the path of the radiation and then position a second bowtie filter housed in the carriage in the path of the radiation.

The technical effect of attenuating a beam reaching the subject by using a hardening filter is that a higher-powered x-ray source with increased x-ray tube temperature may be used during a scan without increasing radiation exposure of the subject. Overall, the higher power improves the quality of the diagnostic scan and improves thermal stability of the x-ray tube including the target.

In one example, an imaging system, includes a carriage with one or more hardening filters and one or more bowtie filters. A filter driving system moves carriage to selectively position the one or more hardening filters and the one of the one or more bowtie filters in a path of a radiation beam between a radiation source and an imaging subject. The one or more hardening filters extends from an edge of the carriage and may overlap with a bowtie filter in a second carriage. The one or more hardening filters extending from the carriage may be coupled to the carriage using a support structure. In the preceding example method, additionally or optionally, one or more hardening filter may partially overlap with the one or more bowtie filters. In any or all of the preceding examples, additionally or optionally, the one or more bowtie filters include a first bowtie filter, and a second bowtie placed adjacent to each other within the carriage. In any or all of the preceding examples, additionally or optionally, a hardening filter is placed between the first bowtie filter and the second bowtie filter, the hardening filter partially overlapping with each of the first bowtie filter and the second bowtie filter. In any or all of the preceding examples, additionally or optionally, the first bowtie filter is housed within a first slot formed in a cavity of the carriage and wherein the second bowtie filter is housed within a second slot formed in the cavity of the carriage, the first slot separated from the second slot via a tab. In any or all of the preceding examples, additionally or optionally, the hardening filter is embedded within a recess between the first bowtie filter and the second bowtie filter, the one or more hardening filters coupled to the tab. In any or all of the preceding examples, additionally or optionally, each of the hardening filter includes a support structure and one or more metallic sheets, the support structure and the one or more metallic sheets stacked together and coupled to the tab via a plurality of bolts. In any or all of the preceding examples, additionally or optionally, the support structure and the one or more metallic sheets may be of a same dimension, the support structure made of a material different from that of the one or more metallic sheets.

In any or all of the preceding examples, additionally or optionally, the radiation beam passed through the one or more hardening filters, then one of the one or more bowtie filters, and the aluminum filter prior to entering the imaging subject. In any or all of the preceding examples, additionally or optionally, the filter driving system includes a motor coupled to the carriage via a shaft, the motor operated to translate the shaft for positioning the one or more hardening filters and the one of the one or more bowtie filters in the path.

Another example method for an imaging system includes, during a first imaging, moving a carriage to position a hardening filter and a first bowtie filter housed in the carriage in a path of a radiation beam between a radiation source and an imaging subject, and during a second imaging, moving the carriage to move the hardening filter and the first bowtie filter out of the path of the radiation and then position a first bowtie filter or a second bowtie filter housed in the carriage in the path of the radiation. In the preceding example method, additionally or optionally, the first imaging is a scout scan and a second imaging is a diagnostic scan of an anatomy of the imaging subject, a beam size used in the first imaging smaller than a beam size used in the second imaging. In any or all of the preceding examples, additionally or optionally, the moving the carriage includes actuating a motor coupled to the carriage via a shaft, the shaft translating in a direction perpendicular to a direction of the path of the radiation to position one or more of the hardening filters, the first bowtie filter, and the second bowtie filter in the path of the radiation. In any or all of the preceding examples, additionally or optionally, each of the first bowtie filter and the second bowtie filter are positioned inside corresponding, adjacent slots within the carriage and the hardening filter is coupled to the carriage between the first bowtie filter and the second bowtie filter. In any or all of the preceding examples, additionally or optionally, the hardening filter partially overlaps with each of the first bowtie filter and the second bowtie filter, and herein, during the first imaging, the radiation beam first passes through the hardening filter and then passes through the first bowtie filter. In any or all of the preceding examples, additionally or optionally, one or more hardening filters extends from a first carriage and may be moved to overlap a bowtie filter positioned within a second carriage.

In yet another example, a system for an imaging system, includes a gantry for receiving an imaging subject, a radiation source positioned in the gantry for emitting radiation exposure, a detector positioned on the opposite of the gantry relative to the radiation source, a motorized table for moving the imaging subject within the gantry, a computation device with instructions stored in a non-transient memory, a one or more bowtie filters, and one or more hardening filters positioned in the filter carriage. The one or more hardening filters may extend from the carriage. In some examples, the one or more hardening filter may overlap with a bowtie filter of a second carriage. Additionally or alternatively, the one or more hardening filters may be mounted in between a first bowtie filter of the one or more bowtie filters and a second bowtie filter of the one or more filters, partially overlapping with each of the first bowtie filter and the second bowtie filter. A filter driving system for switching filters by moving one or more of the bowtie filters, and/or the one or more hardening filters into or out of the radiation beam. In the preceding example system, additionally or optionally, each of the bowtie filters includes a first, straight long side and a second, parallel long side including a central ridge. Each of the bowtie filters is made of graphite. In any or all of the preceding examples, additionally or optionally, the hardening filter includes each of a rectangular support structure, and one or more rectangular metallic sheets stacked under the support structure. In any or all of the preceding examples, additionally or optionally, the rectangular support structure is made of aluminum and the one or more rectangular metallic sheets are made of copper with each of the one or more rectangular metallic sheets having a different thickness.

FIGS. 3-16F show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An imaging system, comprising:
an x-ray source to generate an x-ray beam;
a pre-patient collimator positioned adjacent to the x-ray source such that the x-ray beam passes through the pre-patient collimator, wherein the pre-patient collimator includes:
a carriage; and
at least one filter coupled to one edge of the carriage and extending away from the carriage.

2. The system of claim 1, wherein the carriage further includes a bowtie filter positioned within the carriage.

3. The system of claim 1, wherein the carriage is a first carriage, further comprising a second carriage wherein the second carriage includes one or more bowtie filters, and wherein one of the at least one filter of the first carriage may overlap with one of the one or more bowtie filters of the second carriage when the first carriage and the second carriage are positioned such that one of the at least one filter and one of the one or more bowtie filters are in the path of the x-ray beam.

4. The system of claim 3, wherein the at least one filter is coupled to a top surface of the edge of the first carriage such that the at least one filter may be positioned above one of the one or more bowtie filters of the second carriage to overlap the one or more bowtie filters.

5. The system of claim 3, wherein the x-ray beam passes through the at least one filter, then one of the one or more bowtie filters prior to entering an imaging subject.

6. The system of claim 1, further including a filter driving system for moving the carriage to selectively position one of the at least one filter of the carriage in a path of the x-ray beam between the x-ray source and an imaging subject.

7. The system of claim 6, wherein the filter driving system includes a motor coupled to the carriage via a shaft, the motor operated to translate the shaft for positioning one of the at least one filter in the path of the x-ray beam.

8. The system of claim 1, wherein the at least one filter is a heavy element filter that emits mono-energy x-ray beam for energy calibration and is used in detector energy calibration.

9. The system of claim 1, wherein the at least one filter includes a support structure and one or more metallic sheets, the support structure and the one or more metallic sheets stacked together and coupled to the at least one filter via a plurality of fasteners.

10. The system of claim 9, wherein the support structure and the one or more metallic sheets may be of a same dimension, and wherein the support structure made of a material different from that of the one or more metallic sheets.

11. A filter assembly for a pre-patient collimator assembly to be used with an imaging device, the filter assembly comprising:
a carriage;
a first filter coupled to a top edge of the carriage, wherein the first filter extends away from the top edge;
a frame member to support the first filter; and
a second filter positioned within a slot of the carriage, wherein a top surface of the second filter does not extend past the top edge of the carriage.

12. The filter assembly of claim 11, wherein the frame member includes a first window, wherein the first filter is positioned within the first window.

13. The filter assembly of claim 11, wherein the first filter is a heavy element filter that emits mono-energy x-ray beam for energy calibration and is used in detector energy calibration.

14. The filter assembly of claim 13, wherein the first filter is made of at least one of copper, tungsten, aluminum, tin, silver, ytterbium, hafnium, and bismuth, or a combination thereof.

15. The filter assembly of claim 11, further including a third filter coupled to the top edge of the carriage, wherein the third filter is positioned within the frame member adjacent to the first filter.

16. The filter assembly of claim 15, wherein the third filter is a heavy element filter that emits mono-energy x-ray beam for energy calibration and is used in detector energy calibration, and wherein the third filter is made of at least one of copper, tungsten, aluminum, tin, silver, ytterbium, hafnium, and bismuth, or a combination thereof.

17. The filter assembly of claim 16, wherein the third filter has a thickness that is different than a thickness of the first filter.

18. The filter assembly of claim 11, wherein the second filter is a bowtie filter.

19. A pre-patient collimator assembly for an imaging system, the filter assembly comprising:
a first carriage including one or more bowtie filters positioned within the first carriage, and one or more hardening filters coupled to a top edge of the first carriage and extending away from the first carriage;
a first filter driving system including a first motor and a first shaft operative to move the first carriage within the filter assembly;
a second carriage including one or more bowtie filters positioned within the second carriage; and
a second filter driving system including a second motor and a second shaft operative to move the second carriage within the filter assembly, wherein the first and second carriages are oriented within the filter assembly such that the one or more hardening filters of the first carriage may overlap the one or more bowtie filters of the second carriage when the first carriage and the second carriage are moved to at least one position within the filter assembly.

20. The pre-patient collimator assembly of claim 19, wherein each of the one or more bowtie filters of the first and second carriages has a different width.
